(19) United States
(12) Patent Application Publication
(10) Pub. No.: US 2025/0188401 A1
(43) Pub. Date: Jun. 12, 2025

(54) SILICON FILTERS FOR CELL MECHANOPORATION

(71) Applicant: Portal Biotechnologies, Inc., Arlington, MA (US)

(72) Inventors: Alec John BARCLAY, Winchester, MA (US); Armon Reza SHAREI, Arlington, MA (US); Jacquelyn Lee SIKORA HANSON, Sudbury, MA (US); Pedro Alejandro DUARTE RIVEROS, Edmonton (CA); Michael FINOT, Edmonton (CA)

(21) Appl. No.: 18/975,659

(22) Filed: Dec. 10, 2024

Related U.S. Application Data
(60) Provisional application No. 63/608,754, filed on Dec. 11, 2023.

(51) Int. Cl. C12M 1/42

(52) U.S. Cl. CPC C12M 35/04

(57) ABSTRACT

The present disclosure provides silicon filters for cell mechanoporation, methods of manufacturing the silicon cell mechanoporation filters, cell mechanoporation systems comprising the silicon filters, and methods of use thereof. The silicon cell mechanoporation filters described herein may comprise a silicon filtering surface and a plurality of pores extending through the silicon filtering surface that are configured to perturb cell membranes as a cell mixture passes through the plurality of pores. The silicon cell mechanoporation filters described herein may include a support structure disposed on a side of the silicon filtering surface that covers at least a portion of the silicon filtering surface. The support structure may cover at least 1% of the silicon filtering surface and may enable the silicon filter to receive the cell mixture at a volumetric flow rate between 0.5-500 mL/min per mm² porous surface area.

### CROSS-REFERENCE TO RELATED APPLICATIONS

This application claims priority to and the benefit of U.S. Provisional Patent Application No. 63/608,754, filed Dec. 11, 2023, the contents of which are incorporated herein in its entirety.

### FIELD

This disclosure relates generally to cell mechanoporation devices, and more specifically to silicon filters including a porous silicon surface for cell mechanoporation.

### BACKGROUND

Intracellular delivery of macromolecules such as nucleic acids, peptides and proteins is a challenge in research and therapeutic applications. The delivery of the aforementioned materials into cells can be controlled by rapidly mechanically deforming the cell to produce transient cell membrane disruptions that allow the macromolecules to enter the cell cytosol (otherwise referred to herein as mechanoporation). Known techniques of mechanical deformation of cells include the use of constricting microfluidic channels or porous surfaces (e.g., filters) that cause perturbations in the cell membranes. These microfluidic channels and filters have been introduced to large cell processing systems for intracellular delivery. However, the design and fabrication of the filters and channels is dependent on several parameters that in combination have been challenging to ascertain. Thus, known filters and channels continue to face problems including clogging over time and exhibiting failure (e.g., breakage) at the pressure and flow rate conditions necessary for efficacious cell mechanoporation, thereby precluding widespread adaption and use of these devices.

### SUMMARY

Described herein are silicon filters for cell mechanoporation, including methods of use thereof, methods of fabrication, and systems including the silicon cell mechanoporation filters. The silicon cell mechanoporation filters described herein can include a porous silicon filtering surface for perturbing cell membranes and a support structure disposed on a side of the silicon filtering surface. The support structure can cover at least a portion of the silicon filtering surface, thereby enabling the silicon cell mechanoporation filters described herein to withstand the flow and pressure conditions induced by compatible cell processing systems. For example, using the silicon cell mechanoporation filters described herein, cell mixtures can be passed through the pores of the silicon filtering surface at volumetric flow rates between about 0.5-500 mL/min per mm² porous surface area. The silicon cell mechanoporation filters may be configured to receive a cell mixture having a volume of at least about 100 mL and comprising at least about 100 million cells. The silicon cell mechanoporation filters may be configured to pass this number of cells in a single run without replacement and in a short amount of time (e.g., under 30 minutes). Moreover, cell mechanoporation using the silicon filters described herein can be performed without the filter breaking or rapidly clogging. In turn, the silicon cell mechanoporation filters described herein can be integrated to a variety of cell processing systems, including research-scale, clinical scale, and high-throughput screening (HTS) systems to enable intracellular delivery for downstream cell therapy applications with the transfected cell mixture. The described silicon filters may otherwise be referred to herein as silicon chip filters. The silicon filters may be formed from silicon wafers.

In some examples, a silicon filter for cell mechanoporation is provided, comprising: a silicon filtering surface; a plurality of pores extending through the silicon filtering surface and configured to perturb cell membranes as a cell mixture passes through the plurality of pores; and a support structure disposed on a side of the silicon filtering surface and covering at least 1% of the silicon filtering surface.

In some examples, a method for intracellular delivery of a payload is provided, comprising: passing a cell mixture comprising cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface and covering at least 1% of the silicon filtering surface; and collecting a perturbed cell mixture comprising the cells with the payload in the cells.

In some examples, a cell mechanoporation system is provided, comprising: a filter holder fluidly connectable to a cell mixture source; one or more silicon cell mechanoporation filters disposed in the filter holder, the one or more silicon cell mechanoporation filters comprising: a silicon filtering surface; a plurality of pores extending through the silicon filtering surface and configured to perturb cell membranes as a cell mixture from the cell mixture source passes through the plurality of pores; and a support structure disposed on a side of the silicon filtering surface and covering at least 1% of the silicon filtering surface; an output reservoir fluidly connected to the filter holder to collect a perturbed cell mixture; and a pump configured to move the cell mixture through the one or more silicon cell mechanoporation filters and into the output reservoir.

In some examples, a method of fabricating a silicon cell mechanoporation filter, comprising: creating a plurality of pores in a filtering layer of a silicon wafer, the silicon wafer comprising a support layer, an oxide layer on a side of the support layer, and the filtering layer on a side of the oxide layer opposite the support layer; creating a support structure in the support layer of the silicon wafer, wherein the support structure covers at least 1% of the filtering layer; and removing at least a portion of the oxide layer to expose the plurality of pores in the filtering layer and create one or more silicon cell mechanoporation filters.

In some examples, a method for intracellular delivery of a payload, comprising: passing a cell mixture comprising at least 1.0×10⁸ cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface that covers at least a portion of the silicon filtering surface; and collecting a perturbed cell mixture comprising the cells with the payload in the cells.

### BRIEF DESCRIPTION OF THE FIGURES

The present application can be best understood by reference to the following description taken in conjunction with the accompanying figures included in the specification.

FIG. 1A illustrates an exemplary cell mechanoporation filter, in accordance with some embodiments.

FIG. 1B illustrates a close-up view of a portion of the exemplary cell mechanoporation filter, in accordance with some embodiments.

FIG. 1C illustrates a close-up view of the pores of the exemplary cell mechanoporation filter, in accordance with some embodiments.

FIG. 2A illustrates an exemplary cell mechanoporation filter schematic, in accordance with some embodiments.

FIG. 2B illustrates another exemplary cell mechanoporation filter schematic, in accordance with some embodiments.

FIG. 2C illustrates another exemplary cell mechanoporation filter schematic, in accordance with some embodiments.

FIG. 2D illustrates another exemplary cell mechanoporation filter schematic, in accordance with some embodiments.

FIG. 2E illustrates another exemplary cell mechanoporation filter schematic, in accordance with some embodiments.

FIG. 2F illustrates another exemplary cell mechanoporation filter schematic, in accordance with some embodiments.

FIG. 2G illustrates another exemplary cell mechanoporation filter schematic, in accordance with some embodiments.

FIG. 2H illustrates another exemplary cell mechanoporation filter schematic, in accordance with some embodiments.

FIG. 2I illustrates another exemplary cell mechanoporation filter schematic, in accordance with some embodiments.

FIG. 2J illustrates another exemplary cell mechanoporation filter schematic, in accordance with some embodiments.

FIG. 2K illustrates another exemplary cell mechanoporation filter schematic, in accordance with some embodiments.

FIG. 2L illustrates another exemplary cell mechanoporation filter schematic, in accordance with some embodiments.

FIG. 3A illustrates a process diagram of an example cell mechanoporation system comprising a silicon cell mechanoporation filter, in accordance with some embodiments.

FIG. 3B illustrates another process diagram of an example cell mechanoporation system comprising a silicon cell mechanoporation filter, in accordance with some embodiments.

FIG. 3C illustrates another process diagram of an example cell mechanoporation system comprising a silicon cell mechanoporation filter, in accordance with some embodiments.

FIG. 4A illustrates a cross-sectional view of an exemplary silicon wafer schematic for fabricating silicon cell mechanoporation filters, in accordance with some embodiments.

FIG. 4B illustrates a cross-sectional view of the exemplary silicon wafer schematic with pores created in a filtering layer of the silicon wafer, in accordance with some embodiments.

FIG. 4C illustrates a top view of the exemplary silicon wafer schematic with pores, in accordance with some embodiments.

FIG. 4D illustrates a cross-sectional view of the exemplary silicon wafer schematic with a support structure created in a support layer of the silicon wafer, in accordance with some embodiments.

FIG. 4E illustrates a top view of the exemplary silicon wafer schematic with the support structure, in accordance with some embodiments.

FIG. 4F illustrates a cross-sectional view of a silicon filter schematic resulting from fabricating the silicon wafer, in accordance with some embodiments.

FIG. 5A illustrates results of an existing cell mechanoporation filter in withstanding pressure conditions at a flow rate of 50 mL/min, in accordance with some embodiments.

FIG. 5B illustrates results of a 5×5 mm filter schematic 200c in withstanding pressure conditions at a flow rate of 50 mL/min, in accordance with some embodiments.

FIG. 5C illustrates results of a 10×10 mm filter schematic 200c in withstanding pressure conditions at a flow rate of 50 mL/min, in accordance with some embodiments.

FIG. 5D illustrates results of the filter schematic 200d in withstanding pressure conditions at a flow rate of 50 mL/min, in accordance with some embodiments.

FIG. 5E illustrates results of the filter schematic 200e in withstanding pressure conditions at a flow rate of 50 mL/min, in accordance with some embodiments.

FIG. 5F illustrates results of the filter schematic 200f in withstanding pressure conditions at a flow rate of 50 mL/min, in accordance with some embodiments.

FIG. 5G illustrates results of the filter schematic 200g in withstanding pressure conditions at a flow rate of 50 mL/min, in accordance with some embodiments.

FIG. 5H illustrates results of the filter schematic 200h in withstanding pressure conditions at a flow rate of 50 mL/min, in accordance with some embodiments.

FIG. 5I illustrates results of the filter schematic 200i in withstanding pressure conditions at a flow rate of 50 mL/min, in accordance with some embodiments.

FIG. 5J illustrates results of the filter schematic 200j in withstanding pressure conditions at a flow rate of 50 mL/min, in accordance with some embodiments.

### DETAILED DESCRIPTION

Silicon cell mechanoporation filters, systems, methods of use thereof, and methods of fabrication thereof are described herein. The silicon cell mechanoporation filters can include a silicon filtering surface and a support structure on a side of the silicon filtering surface covering at least a portion of the filtering surface. The silicon filtering surface can include a plurality of pores extending therethrough for mechanically deforming cells as a cell mixture is passed through the silicon filter, thereby causing perturbation of the cell membranes. The support structure can extend across the silicon filtering surface in at least one direction to cover at least a portion (e.g., 1%) of the silicon filtering surface. The silicon cell mechanoporation filters may be configured to withstand a volumetric flow rate between about 0.5-500 mL/min per mm² porous surface area. The silicon cell mechanoporation filters may be configured to receive a cell mixture having a volume of at least about 100 mL and comprising at least about 100 million cells. Further, the silicon filters described herein may be configured to pass this large quantity of cells in a single run without replacement, clogging, or breakage, and in a short amount of time. The silicon filters described herein can be integrated to or used in conjunction with a variety of cell processing systems, whether it be custom research-scale systems, clinical-scale systems (e.g., filtration systems, cell washing systems, elutriation systems, isolation systems), high-throughput screening systems, etc. Using the silicon filters described herein, membranes of cells from a cell mixture can be perturbed to enable intracellular delivery of a payload to the cell, and the resulting transfected cell mixture can be used in numerous downstream cell therapy applications.

The silicon cell mechanoporation filters described herein can be formed from a silicon wafer that comprises two silicon layers and an oxide layer buried between the two silicon layers. A plurality of pores can be generated in one of the silicon layers of the wafer (i.e., a filtering layer), and a support structure can be generated in the other silicon layer of the wafer (i.e., a support layer). After fabricating the support structure and pores, the buried oxide layer can be removed to expose the plurality of pores in the filtering layer and create one or more silicon cell mechanoporation filters. Generating the pores and the support structure can utilize known photoresist coating and photolithography techniques, such as reactive ion etching, with specific parameters. Several silicon filters can be generated from a single silicon wafer, thus, after removing the oxide layer, the method can include cutting the silicon wafer into a plurality of individual silicon filters.

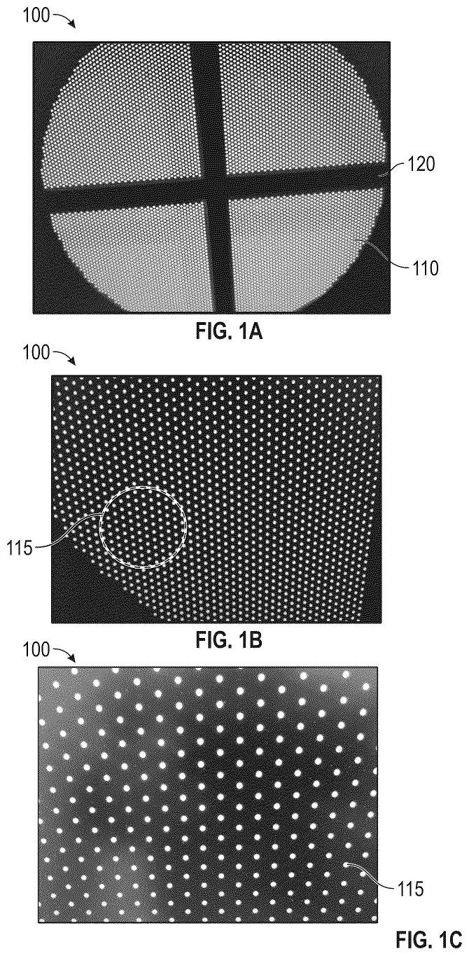

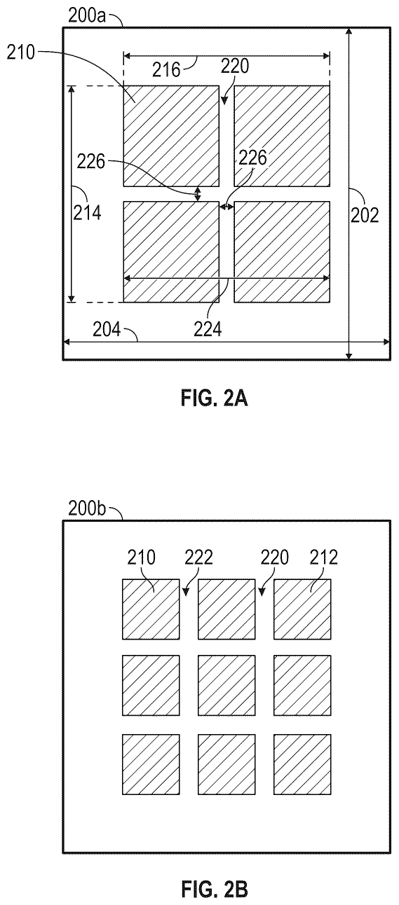

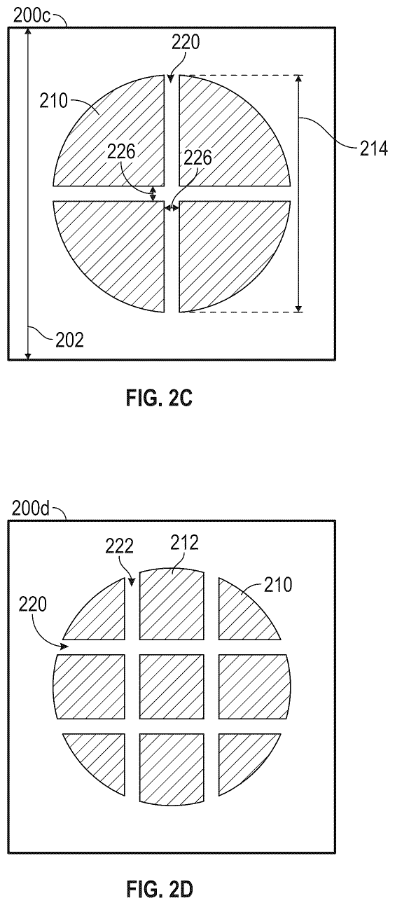

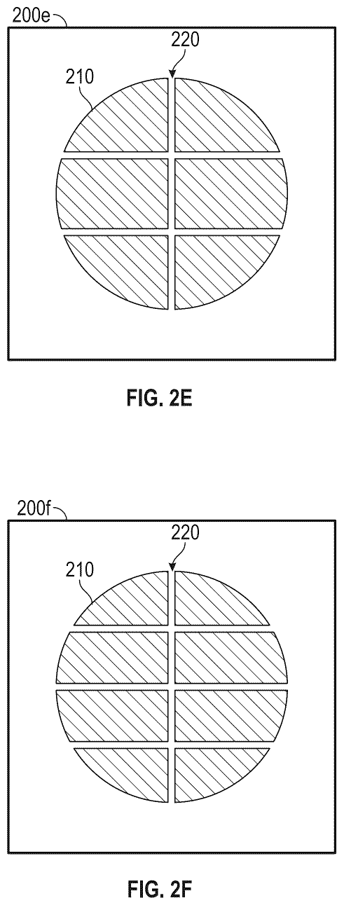

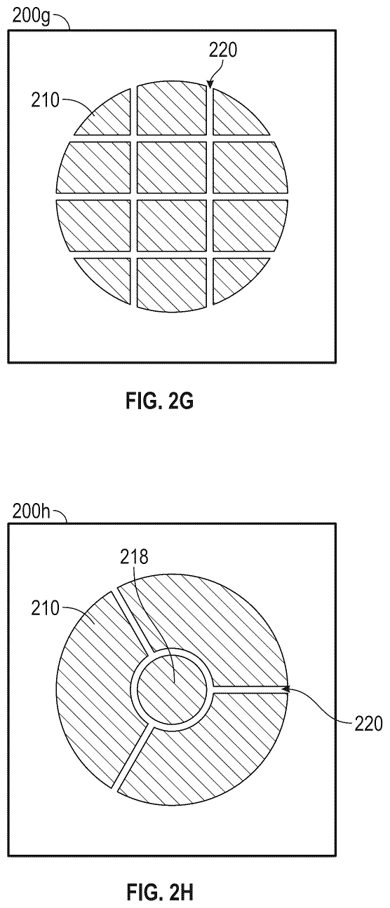

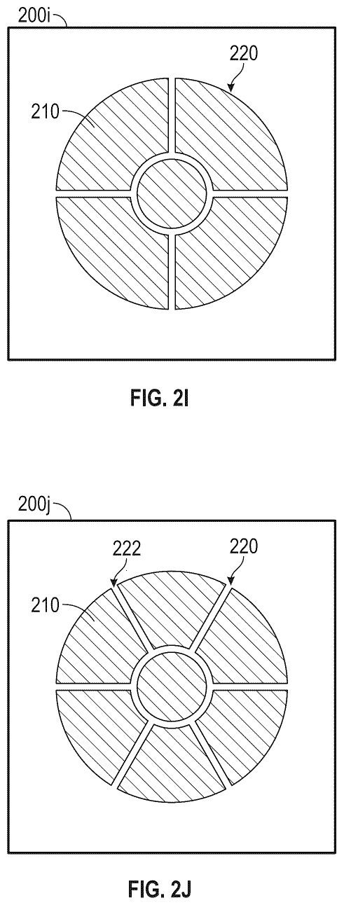

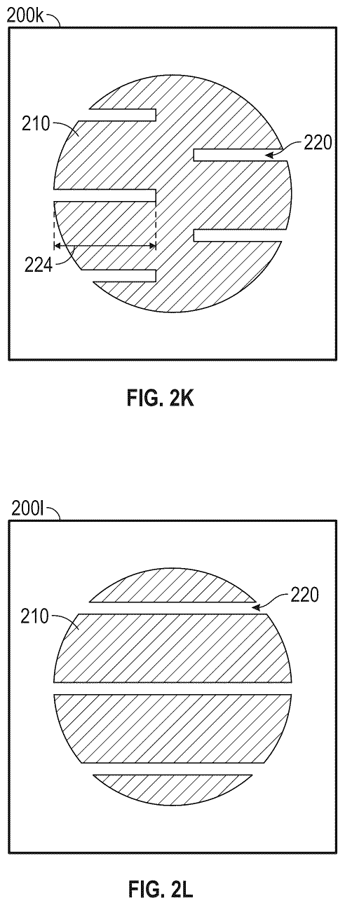

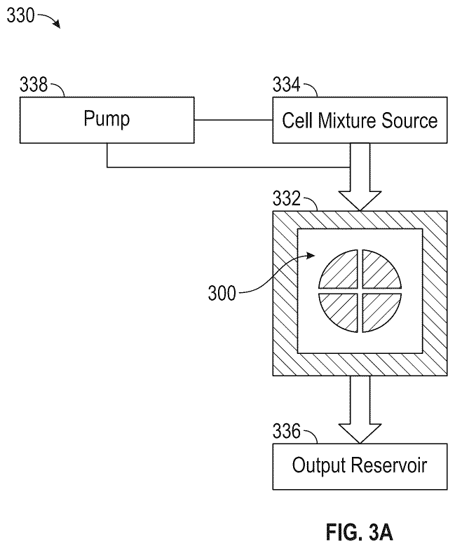

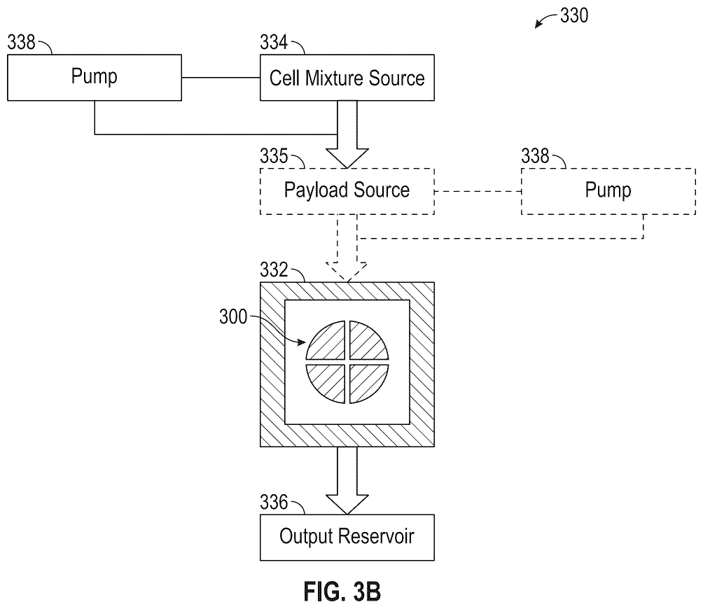

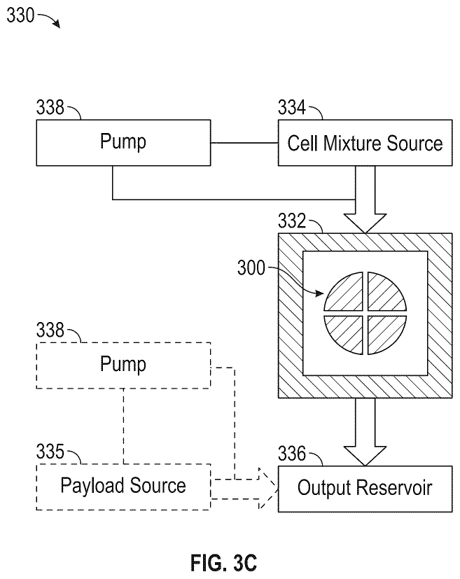

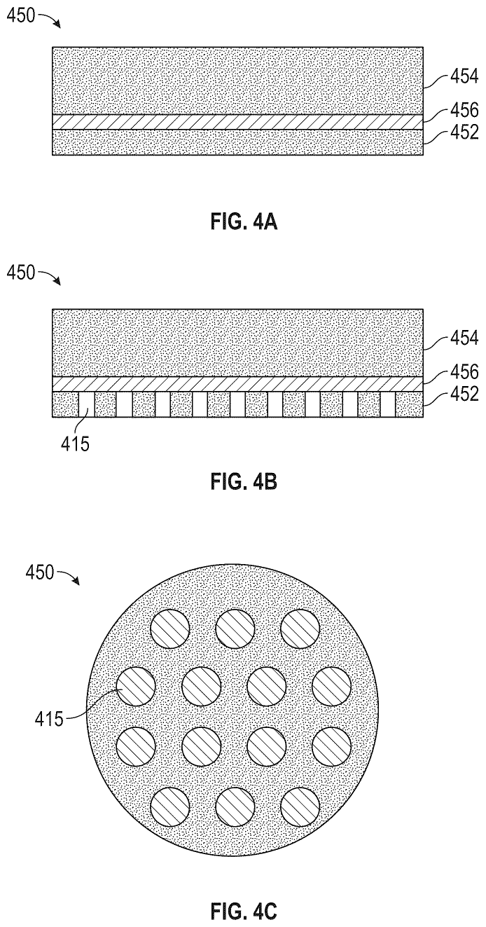

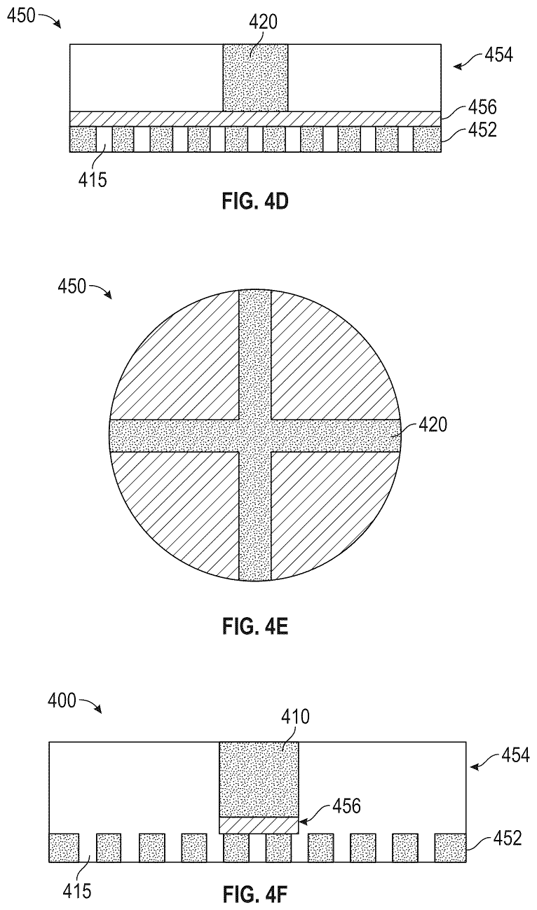

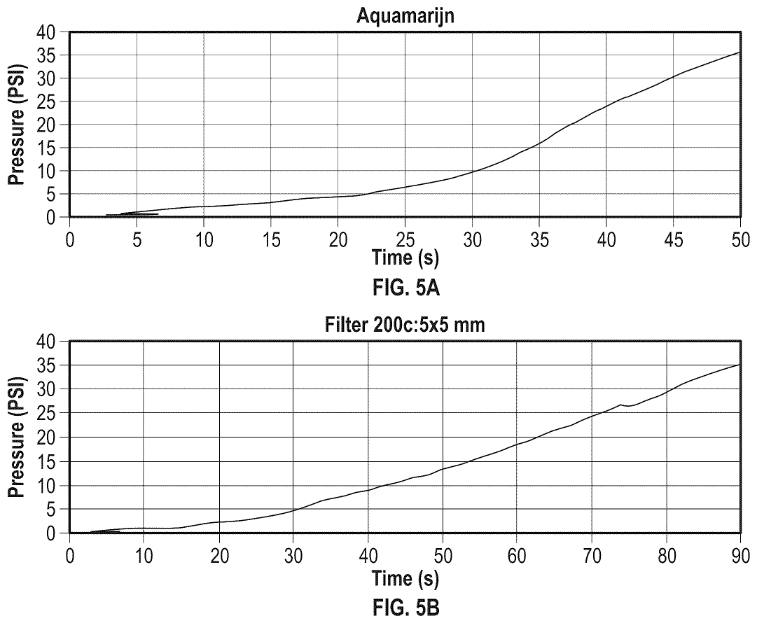

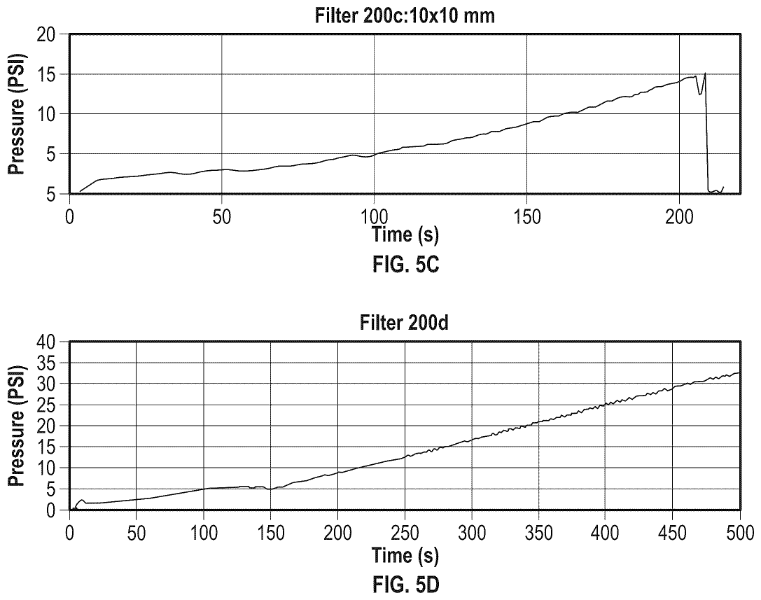

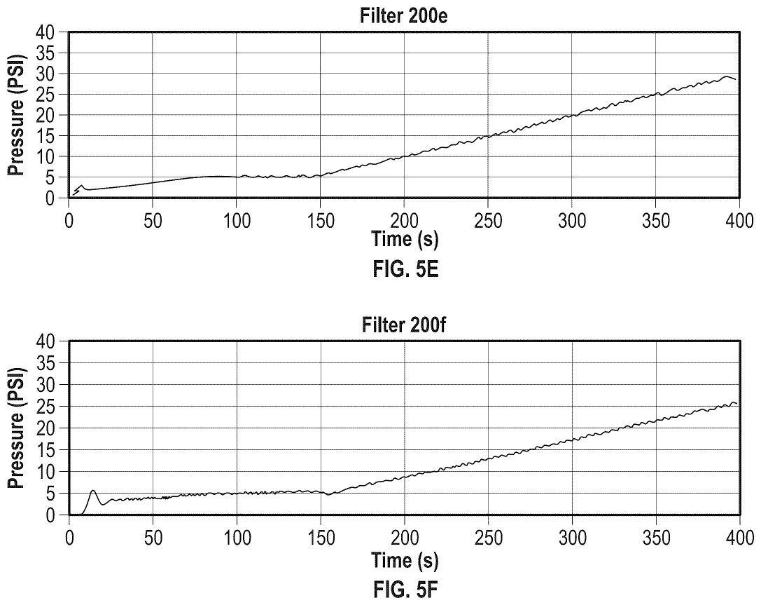

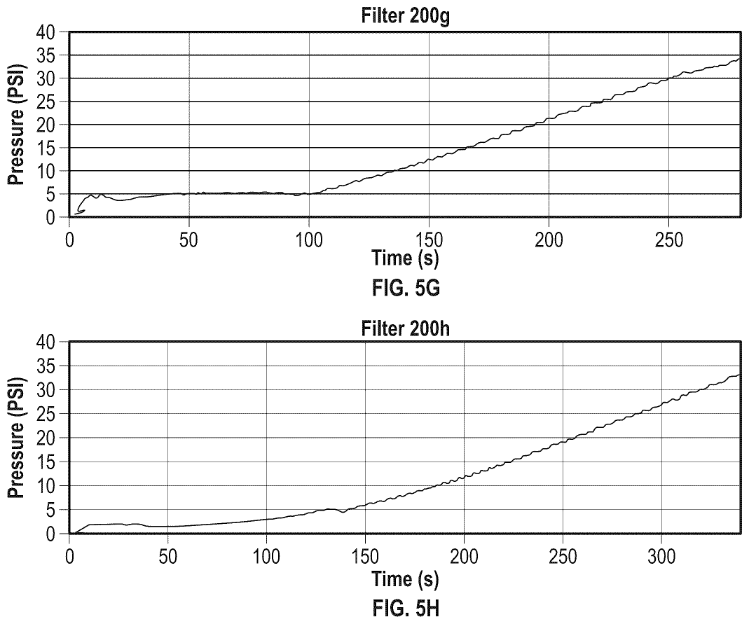

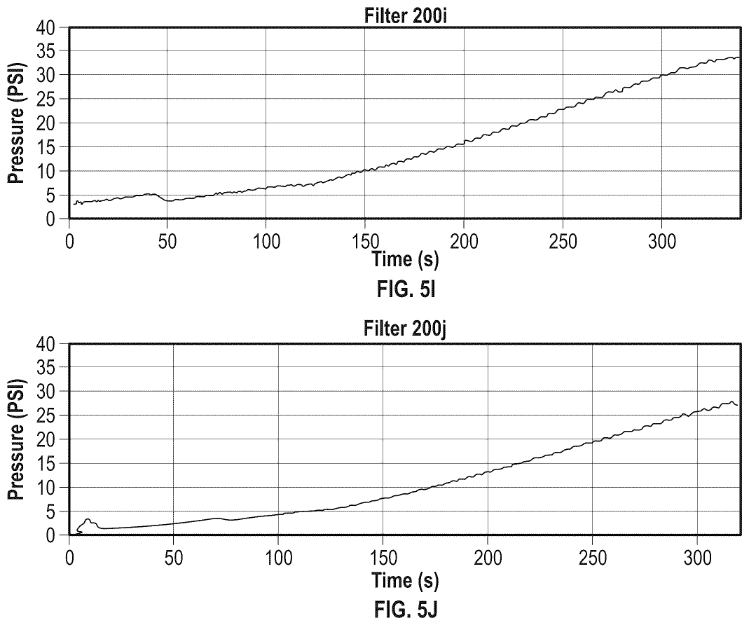

The following disclosure describes example silicon cell mechanoporation filters with reference to various embodiments, illustrated at least in FIGS. 1A-1C and FIGS. 2A-2L. Example cell mechanoporation systems comprising the silicon cell mechanoporation filters provided herein are described with respect to FIGS. 3A-3C. An example method for intracellular delivery of a payload is also described herein. An example method of fabricating the silicon cell mechanoporation filters provided herein is described with respect to FIGS. 4A-4F. Finally, results from testing various filter schematics of the cell mechanoporation filters illustrated in FIGS. 2A-2L are provided herein and described with respect to FIGS. 5A-5J.

### Silicon Cell Mechanoporation Filters

In some embodiments, a silicon filter for cell mechanoporation is provided. FIGS. 1A-1C illustrate a silicon filter 100. The silicon filter 100 may comprise a silicon filtering surface 110 and a support structure 120 disposed on a side of the silicon filtering surface 110. As shown in FIG. 1A, the support structure 120 may cover at least a portion (e.g., 1%) of the silicon filtering surface 110. As shown at least in FIGS. 1B-1C, the filter 100 may comprise a plurality of pores 115 extending through the silicon filtering surface, the pores 115 configured to perturb cell membranes as a cell mixture passes through the plurality of pores 115.

Cell mechanoporation requires a certain flow rate for efficacious cell membrane perturbation. A flow rate that is too low may not adequately perturb the cell membranes. A flow rate that is too high may over-perturb and thus destroy the cell, rendering the sample unusable. In some embodiments, the silicon filter 100 for cell mechanoporation may be configured to receive a volumetric flow rate between about 0.5-500 mL/min per mm² porous surface area. The porous surface area may be defined as the sum of the area of all total pores in the porous filtering surface. For example, the silicon filter 100 may be configured to receive a volumetric flow rate of about 0.5-100, 0.5-10, 50-500, 5-100, or 5-20 mL/min per mm² porous surface area. In some embodiments, the silicon filter 100 may be configured to receive a volumetric flow rate greater than or equal to about 0.5, 1, 2, 2.5, 3, 4, 5, 6, 7, 8, 9, 10, 12, 15, 18, 20, 25, 30, 35, 40, 45, 50, 75, 100, 125, 150, 175, 200, 250, 300, 350, 400, or 450 mL/min per mm² porous surface area. In some embodiments, the silicon filter 100 may be configured to receive a volumetric flow rate less than or equal to about 1, 2, 2.5, 3, 4, 5, 6, 7, 8, 9, 10, 12, 15, 18, 20, 25, 30, 35, 40, 45, 50, 75, 100, 125, 150, 175, 200, 250, 300, 350, 400, 450, or 500 mL/min per mm² porous surface area. In some embodiments, the silicon filter 100 may be configured to receive a volumetric flow rate greater than 500 mL/min per mm² porous surface area, such as about 550, 600, 650, 700, or 750 mL/min per mm² porous surface area. In some embodiments, the silicon filter 100 may be configured to receive a volumetric flow rate less than 0.5 mL/min per mm² porous surface area, such as about 0.1, 0.2, 0.3, or 0.4 mL/min per mm² porous surface area.

In some embodiments, the silicon filter 100 for cell mechanoporation may be configured to receive a constant volumetric flow. A constant volumetric flow may allow the speed of the cells to remain consistent as the cell mixture is passed through the silicon filter 100, thereby improving the performance of cell mechanoporation by the silicon filter 100. Alternatively or additionally, the silicon filter 100 may be configured to receive a pulsatile flow. The flow may be controlled by at least one of a peristaltic pump, a syringe pump, and a pressurized reservoir. In some embodiments, the flow through the silicon filter 100 may be manually controlled, such as by using a hand-operated syringe.

In some examples, the volumetric flow through the silicon filter 100 is controlled by a centrifuge. For example, the silicon filter 100 can be contained in a cartridge configured to be inserted to a centrifuge, and the centrifuge can be programmed for mechanoporation. Exemplary parameters that may be set on the centrifuge and may affect the silicon filter 100 can include at least relative centripetal force (RCF) and/or gravitational force (G-force). Thus, the silicon filter 100 can be designed in accordance with the variables set forth herein to withstand these parameters and effectively perturb cell membranes based thereon. In some examples, the G-force may be between about 10-10,000 G. For example, the gravitational force may be less than or equal to about 10,000 G, 7,500 G, 5,000 G, 2,500 G, 1,000 G, 500 G, 300 G, or 100 G. The gravitational force may be greater than or equal to about 10 G, 50 G, 100 G, 250 G, 500 G, 1,000 G, or 2,500 G.

In some embodiments, the RCF may be between about 20-800 RCF. For example, the RCF may be less than or equal to about 800 RCF, 750 RCF, 700 RCF, 650 RCF, 600 RCF, 550 RCF, 500 RCF, 450 RCF, 400 RCF, 350 RCF, 300 RCF, 250 RCF, 200 RCF, 180 RCF, 130 RCF, 110 RCF, 100 RCF, 80 RCF, 60 RCF, or 50 RCF. In some embodiments, the RCF may be greater than or equal to about 20 RCF, 50 RCF, 60 RCF, 80 RCF, 100 RCF, 110 RCF, 130 RCF, 180 RCF, 200 RCF, 250 RCF, 300 RCF, 350 RCF, 400 RCF, 450 RCF, 500 RCF, 550 RCF, 600 RCF, 650 RCF, 700 RCF, or 750 RCF. The silicon filter 100 may mechanically disrupt cells in accordance with these parameters while retaining cell viability. Cell mechanoporation using a centrifuge system is described in greater detail in PCT/US2024/038369, the contents of which are incorporated herein in its entirety.

The silicon filter 100 may be usable within or downstream of various cell processing systems that process large fluid quantities (i.e., clinical-scale systems). Additionally or alternatively, the silicon filter 100 may be integrated to research-scale systems that process smaller quantities of fluid. Thus, the silicon filter 100 may be suited to receive a wide range of fluid quantities. The silicon filter 100 may be configured to receive a large range of volumes, such as between at least 10 μL and 1000 L (e.g., about 100 mL). In some embodiments, the silicon filter 100 may be configured to receive a volume between about 1-1000 μL, 1-100 μL, 1-1000 mL, 1-100 mL, or 1-1000 L. For example, the silicon filter 100 may be configured to receive a volume greater than or equal to about 10 μL, 25 μL, 50 μL, 75 μL, 100 μL, 150 μL, 200 μL, 250 μL, 300 μL, 350 μL, 400 μL, 450 μL, 500 μL, 600 μL, 700 μL, 800 μL, 900 μL, 1 mL, 2 mL, 3 mL, 4 mL, 5 mL, 10 mL, 15 mL, 20 mL, 25 mL, 30 mL, 40 mL, 50 mL, 75 mL, 100 mL, 250 mL, 500 mL, 750 mL, 1 L, 2 L, 5 L, 10 L, 50 L, 100 L, or 500 L. In some embodiments, the silicon filter 100 may be configured to receive a volume less than or equal to about 25 μL, 50 μL, 75 μL, 100 μL, 150 μL, 200 μL, 250 μL, 300 μL, 350 μL, 400 μL, 450 μL, 500 μL, 600 μL, 700 μL, 800 μL, 900 μL, 1 mL, 2 mL, 3 mL, 4 mL, 5 mL, 10 mL, 15 mL, 20 mL, 25 mL, 30 mL, 40 mL, 50 mL, 75 mL, 100 mL, 250 mL, 500 mL, 750 mL, 1 L, 2 L, 5 L, 10 L, 50 L, 100 L, 500 L. or 1000 L.

Upstream cell processing devices, and more generally, the integration of the silicon filters 100 described herein in cell mechanoporation systems, may induce pressure on the silicon filter 100. As the silicon filter 100 includes a fragile, porous surface for cell mechanoporation, it is important that the silicon filter 100 is formed to withstand various pressure conditions. In some embodiments, the silicon filter 100 for cell mechanoporation may be configured to withstand a pressure of between at least about 1-50 psi. For example, the silicon filter 100 may be configured to withstand a pressure of about 1-25, 1-10, 5-50, 5-25, 10-50, 10-25, or 25-50 psi. In some embodiments, the silicon filter 100 may be configured to withstand a pressure of greater than or equal to about 1, 2, 4, 6, 8, 10, 12, 14, 15, 16, 18, 20, 22, 25, 28, 30, 35, 40, or 45 psi. In some embodiments, the silicon filter 100 may be configured to withstand a pressure of less than or equal to about 2, 4, 6, 8, 10, 12, 14, 15, 16, 18, 20, 22, 25, 28, 30, 35, 40, 45, or 50 psi. In some embodiments, the silicon filter 100 may be configured to withstand a pressure greater than 50 psi, such as about 55, 60, 65, 70, or 75 psi.

The silicon filter 100 may comprising several hundred, thousand, or tens of hundreds (or thousand) pores 115 for perturbing cell membranes. For example, the silicon filter 100 may comprise between about 500-400,000 pores, 500-100,000 pores, 10,000-400,000 pores, 10,000-100,000 pores, 50,000-400,000 pores, 50,000-100,000 pores, or 100,000-400,000 pores. In some embodiments, the silicon filter 100 may comprise at least about 500, 1,000, 5,000, 10,000, 50,000, 75,000, 100,000, 125,000, 150,000, 175,000, 200,000, 225,000, 250,000, 275,000, 300,000, 325,000, 350,000, or 375,000 pores. In some embodiments, the silicon filter 100 may comprise no more than 1,000, 5,000, 10,000, 50,000, 75,000, 100,000, 125,000, 150,000, 175,000, 200,000, 225,000, 250,000, 275,000, 300,000, 325,000, 350,000, 375,000, or 400,000 pores.

The plurality of pores 115 extending through the silicon filtering surface 110 may comprise uniformly-sized pores or varying pore sizes throughout the surface. Different sized pores may lend to varying efficacies of perturbing cell membranes of the different types of cells that may be passed through the plurality of pores 115. The plurality of pores 115 may comprise one or more cross-sectional shapes including but not limited to circular, rectangular (e.g., square), elliptical, triangular, or another polygonal shape. Because the silicon filtering surface 110 exhibits a thickness (described in greater detail below), it is understood that the plurality of pores 115 can exhibit a three-dimensional shape (e.g., a prism) extending through the silicon filtering surface 110. For example, in the instance a pore comprises a circular cross-section with a uniform diameter along the thickness of the filtering surface 110, the 3-dimensional shape of the pore may be a cylinder. In the instance the pore comprises a triangular shape, the 3D shape of the pore may be a triangular prism. In the instance the width of the pore varies (e.g., increases or decreases) along the thickness of the filtering surface 110, the 3D shape of the pore can be conical.

In some embodiments, a width, or size, of each pore of the plurality of pores 115 may be between 2 μm and 20 μm. The width of the pore may be dependent on the type of cell(s) in the cell mixture passed through the plurality of pores 115. For example, the width may be between about 2-15 μm, 2-10 μm, 2-7 μm, 8-15 μm, 8-10 μm, or about 11-15 μm. In some embodiments, the pore width may be greater than or equal to about 2, 2.5, 3, 4, 4.5, 5, 5.5, 6, 6.5, 7, 7.5, 8, 9, 10, 11, 12, 14, 15, 16, or 18 μm. In some embodiments, the pore width may be less than or equal to about 2.5, 3, 4, 4.5, 5, 5.5, 6, 6.5, 7, 7.5, 8, 9, 10, 11, 12, 14, 15, 16, 18, or 20 μm. In some embodiments, each pore of the plurality of pores 115 may comprise a circular cross-section, and the width of the pore may be a diameter of the pore.

In some examples, the plurality of pores 115 are uniformly sized. The uniformity of the pores 115 may be critical to ensure uniform flow rates and overall performance of the filter. For example, the pores may be within about 25% of one another in width (e.g., size). This dimension can otherwise be referred to as the tolerance of the pores. In some examples, the width of the pores may have a tolerance of about 1-20%, 1-15%, 1-10%, or 1-5%. In some examples, the width of pores have a tolerance of greater than or equal to about 0.1%, 0.2%, 0.5%, 1%, 2%, or 5%. In some examples, the width of the pores have a tolerance of less than or equal to about 1%, 2%, 4%, 5%, 8%, 10%, 12%, 15%, 18%, 20%, 22%, or 25%. This tolerancing may only be achievable with silicon, variants of silicon, or other similar materials.

As mentioned above, the width of a given pore in the silicon filter 100 may be dependent on the cell type being passed through the plurality of pores. For example, the width of each of the pores may be smaller than the diameter of the cells in the mixture passing through the silicon filter 100, such that forcing the cell through the pore under pressure causes a perturbation in the membrane of the cell as the cell is constricted by the pore. For example, the pore width may be between 10% and 99% of the diameter of the cells in a cell suspension, such as about 10%, 20%, 30%, 40%, 50%, 60%, 70%, 80%, 90%, 95%, 98%, or 99% of the diameter of the cells. It is to be understood that reference to a diameter of a cell is intended as the diameter of the cell in the cell mixture prior to being passed through the filter, e.g., as the cell approaches the filter, unless otherwise specified.

In some embodiments, a pitch between each pore of the plurality of pores 115 may be between 0.5:1 and 100:1 relative to the width (i.e., size) of the pore. The pitch of the pore may be selected to allow the cell mixture to flow through the silicon filter 100 without backing up, while also maintaining the structure and ability of silicon filter 100 to withstand various flow rate and pressure conditions. In some embodiments, the pitch between pores may be between about 0.5:1 and 5:1, 0.5:1 and 10:1, 0.5:1 and 25:1, or 0.5:1 and 50:1 relative to the width of the pore. In some embodiments, the pitch between pores may be greater than or equal to about 0.5:1, 0.75:1, 1:1, 2:1, 3:1, 4:1, 5:1, 10:1, 12:1, 15:1, 16:1, 18:1, 20:1, 22:1, 24:1, 25:1, 26:1, 28:1, 30:1, 33:1, 36:1, 40:1, 44:1, 46:1, 48:1, 50:1, 75:1, or 100:1. In some embodiments, the pitch between pores may be less than or equal to about 0.5:1, 0.75:1, 1:1, 2:1, 3:1, 4:1, 5:1, 10:1, 12:1, 15:1, 16:1, 18:1, 20:1, 22:1, 24:1, 25:1, 26:1, 28:1, 30:1, 33:1, 36:1, 40:1, 44:1, 46:1, 48:1, 50:1, 75:1, or 100:1. In a non-limiting example, a pore width between 2-7 μm may have a 4:1 pitch, meaning a 2-μm pore may be spaced 8 μm apart from another 2-μm pore.

The plurality of pores 115 may cover between about 0.1% and about 60% of the total surface area of the silicon filter 100. In some embodiments, the plurality of pores 115 may comprise between about 1.0×10¹ to about 1.0×10¹⁰ pores per square millimeter of total surface area of the filter 100. The number of pores per square millimeter may otherwise be referred to herein as the density of the pores. The porous surface may in some embodiments comprise between about 1.0×10¹ to about 1.0×10¹⁵ pores/mm² total surface area of the filter 100. For example, the porous surface may comprise between about 1.0×10¹ to about 1.0×10⁶ pores/mm², 1.0×10³ to about 1.0×10⁶ pores/mm², or about 1.0×10⁴ to about 1.0×10⁶ pores/mm². In some examples, the porous surface may comprise greater than or equal to about 1.0×10¹ pores/mm², 1.0×10² pores/mm², 1.0×10³ pores/mm², 1.0×10⁴ pores/mm², 1.0×10⁵ pores/mm², 1.0×10⁶ pores/mm², 1.0×10⁸ pores/mm², 1.0×10¹⁰ pores/mm², 1.0×10¹² pores/mm², or 1.0×10¹⁴ pores/mm². In some examples, the porous surface may comprise less than or equal to about 1.0×10² pores/mm², 1.0×10³ pores/mm², 1.0×10⁴ pores/mm², 1.0×10⁵ pores/mm², 1.0×10⁶ pores/mm², 1.0×10⁸ pores/mm², 1.0×10¹⁰ pores/mm², 1.0×10¹² pores/mm², 1.0×10¹⁴ pores/mm², or 1.0×10¹⁵ pores/mm².

In some embodiments, each pore of the plurality of pores 115 may extend linearly through the silicon filtering surface 110. For example, the pores may extend through the silicon filter 100 in a direction collinear with the thickness dimension of the silicon filter 100. In some embodiments, the linear extension of the pores 115 through the silicon filtering surface 110 may be collinear with the direction of flow of the cell mixture through the pores 115.

In some embodiments, a thickness of the silicon filtering surface 110 may be between 0.1 μm and 100 μm. As mentioned above, the thickness of the silicon filtering surface 110 may be defined by the axis along which the cell mixture travels as it passes through the silicon filter 100 (e.g., through the plurality of pores 115). The thickness of silicon filtering surface 110 may consider the pore length necessary to perturb the cell membrane while minimizing clogging within the pores. Also, as described herein with respect to other characteristics of the silicon filter 100, the thickness of silicon filtering surface 110 may be selected to withstand breakage during use. In some embodiments, the thickness of the silicon filtering surface 110 may be between about 0.1-10 μm, 1-100 μm, 1-10 μm, or 10-100 μm. In some embodiments, the thickness of the silicon filtering surface 110 may be greater than or equal to about 0.1, 0.5, 1, 2, 5, 8, 10, 12, 15, 18, 20, 25, 50, 75, or 100 μm. In some embodiments, the thickness of the silicon filtering surface 110 may be less than or equal to about 0.1, 0.5, 1, 2, 5, 8, 10, 12, 15, 18, 20, 25, 50, 75, or 100 μm. In some embodiments, the thickness of the silicon filtering surface 110 is about 10 μm.

In some embodiments, a thickness of the support structure 120 may be between 20 μm and 1 mm. Similar to the silicon filtering surface 110, the thickness of the support structure 120 may be defined by the axis along which the cell mixture travels as it passes through the silicon filter 100 (e.g., through the plurality of pores 115). The thickness of support structure 120 may be selected such that the support structure 120 enables the silicon filter 100 to withstand breakage during use, but also does not impede on the flow of the cell mixture through the pores of the silicon filtering surface 110. In some embodiments, the thickness of the support structure 120 may be between about 20 μm-100 μm, 50 μm-1 mm, 50 μm-100 μm, 0.1-1 mm, 0.1-0.5 mm, or 0.5-1 mm. In some embodiments, the thickness of the support structure 120 may be greater than or equal to about 20 μm, 50 μm, 80 μm, 0.1 mm, 0.2 mm, 0.4 mm, 0.5 mm, 0.8 mm, or 1 mm. In some embodiments, the thickness of the support structure 120 may be less than or equal to about 20 μm, 50 μm, 80 μm, 0.1 mm, 0.2 mm, 0.4 mm, 0.5 mm, 0.8 mm, or 1 mm.

The silicon filtering surface 110 may comprise a circular, triangular, rectangular, elliptical, or other polygonal shape. For example, as shown at least in FIGS. 2A-2B, the filtering surface 210, which is understood to be equivalent to filtering surface 110, can include a rectangular (e.g., square) shape. The shape of the silicon filtering surface 110 may be selected to correspond with upstream fluid inlets and/or downstream fluid outlets. The width, or size, of the silicon filtering surface 210 in FIG. 2A is indicated by reference numeral 214. As shown, the width 214 extends from a first side to a second side of the silicon filtering surface 210 opposite the first side. In some embodiments, the width of the silicon filtering surface 210 (and 110) may be between 1 mm and 10 cm. For example, the width of the silicon filtering surface 210 may be between about 1 mm-5 cm, 1 mm-15 mm, 1 mm-1 cm, 1 mm-5 mm, 5 mm-10 cm, 5 mm-1 cm, 1 cm-10 cm, 1 cm-5 cm, or 5 cm-10 cm. In some embodiments, the width of the silicon filtering surface 210 may be greater than or equal to 1 mm, 2 mm, 3.5 mm, 5 mm, 7 mm, 8 mm, 9 mm, 1 cm, 1.1 cm, 2 cm, 5 cm, 8 cm, or 10 cm. In some embodiments, the width of the silicon filtering surface 210 may be less than or equal to 1 mm, 2 mm, 3.5 mm, 5 mm, 7 mm, 8 mm, 9 mm, 1 cm, 1.1 cm, 2 cm, 5 cm, 8 cm, or 10 cm.

The silicon filtering surface 210 (and 110) is described with reference to a single silicon filter 100. However, as mentioned below, a system may in some embodiments comprise a plurality of silicon filters 100. For example, the silicon filters 100 may be arranged in parallel to create an array of silicon filters that together encompass a larger filtering surface. Any of the dimensions provided above with respect to the silicon filtering surface 210 (and 110) are understood to be applicable to this filtering surface composed of a plurality of silicon filters 100. For example, a plurality of silicon filters 100, each silicon filter comprising a width of 1 mm, may be arranged in array to create a silicon filtering surface comprising a width on the magnitude of 1-10 cm.

In some embodiments, the length of the silicon filtering surface 210 (indicated by reference numeral 216 in FIG. 2A) may be substantially the same as the width of the silicon filtering surface 210. In some embodiments, the length of the silicon filtering surface 210 may be greater than or less than the width of the silicon filtering surface 210. Any of the above example ranges of or values for width of the silicon filtering surface 210 are understood to be applicable to the length of the silicon filtering surface 210. In some embodiments, the silicon filtering surface 210 (and 110) can include a circular shape (see, e.g., FIGS. 2C-2J). In this instance, the width may be understood to be the diameter of the silicon filtering surface 210.

In some embodiments, a width of the support structure 120 (e.g., support structure 220) may be at least 30% of the width of the silicon filtering surface 110. As explained above with respect to the width of the silicon filtering surface 110, the width of support structure 120 may extend from a first side to a second side of the silicon filtering surface 110. In FIGS. 2A and 2K, the width of the support structure 220 is indicated by reference numeral 224. The width of support structure 220 may be selected such that the support structure 220 enables the silicon filter 200 (i.e., filters 200a-200l) to withstand breakage during use, but also does not impede on the flow of the cell mixture through the silicon filtering surface 210. In some embodiments, the width of the support structure may be about 30%, 40%, 50%, 60%, 70%, 80%, 90%, 95%, 99%, or may be substantially the same as that of the silicon filtering surface 110. For example, in FIG. 2A, the support structure 220 extends the width of the silicon filtering surface 210. On the other hand, in FIG. 2K, the width of the support structure 220 extends only a portion of the width of the silicon filtering surface 210 (e.g., between about 30-50%).

In some embodiments, a height of the support structure 120 (e.g., support structure 220) may be between 20 μm and 1 mm. The height of the support structure 220 may be defined as extending from a first side to a second side of the support structure 220, indicated in FIG. 2A by reference numeral 226. As mentioned above with respect to the width of the support structure 220, the height of the support structure 220 may be selected such that the support structure 220 enables the silicon filter 200 (filter 200a-200l) to withstand breakage during use, but also does not impede on the cell mixture flow through the pores of the silicon filtering surface 210. In some embodiments, the height of the support structure 120 may be between about 20 μm-0.1 mm, 50 μm-1 mm, 50 μm-0.1 mm, 0.1-1 mm, 0.1-0.5 mm, or 0.5-1 mm. In some embodiments, the height of the support structure 120 may be greater than or equal to about 20 μm, 50 μm, 80 μm, 0.1 mm, 0.2 mm, 0.5 mm, 0.8 mm, or 1 mm. In some embodiments, the height of the support structure 120 may be less than or equal to about 20 μm, 50 μm, 80 μm, 0.1 mm, 0.2 mm, 0.5 mm, 0.8 mm, or 1 mm.

In some embodiments, the support structure 120 may cover at least a portion of the silicon filtering surface 110, such as about 1% of the silicon filtering surface 110. The support structure 120 can cover at least a portion of the silicon filtering surface 110 to support the silicon filtering surface 110 and prevent it from breaking in various flow rate and pressure conditions induced on the filter 100. However, the support structure 120 has to allow the cell mixture to pass through the silicon filtering surface 110 without flow backing up and/or the filtering clogging. Thus, the support structure 120 can cover a portion of the silicon filtering surface 110. In some embodiments, the support structure 120 may cover between about 0.1-30%, 0.1-10%, 0.1-1%, 1-30%, 1-10%, or 1-5% of the silicon filtering surface 110. In some embodiments, the support structure 120 may cover greater than or equal to about 0.1%, 0.2%, 0.5%, 0.8%, 1%, 1.5%, 2%, 2.5%, 3%, 4%, 5%, 6%, 7%, 8%, 9%, 10%, 12%, 15%, 18%, 20%, 22%, 25%, 28%, or 30% of the silicon filtering surface 110. In some embodiments, the support structure may cover less than or equal to about 0.1%, 0.2%, 0.5%, 0.8%, 1%, 1.5%, 2%, 2.5%, 3%, 4%, 5%, 6%, 7%, 8%, 9%, 10%, 12%, 15%, 18%, 20%, 22%, 25%, 28%, or 30% of the silicon filtering surface 110. In some embodiments, the support structure 120 may cover greater than 30% of the silicon filtering surface 110, such as about 35%, 40%, 45%, or 50% of the silicon filtering surface.

As shown in FIG. 1A, the support structure 120 (e.g., support structure 220) may comprise one or more supporting members disposed on the silicon filtering surface 110 and extending in one or more directions. For example, each of the support structures 220 of the silicon filters 200 illustrated in FIGS. 2A-2J comprise a plurality of supporting members 222. As indicated in FIG. 2B, the one or more supporting members 222 can create two or more filtering windows 212 in the silicon filtering surface 210.

As illustrated in FIGS. 2A-2G, the support structure 220 can comprise a plurality of supporting members 222 disposed on the silicon filtering surface 210, and the plurality of supporting members 222 together can form at least one cross shape that separates a plurality of filtering windows 212 in the silicon filtering surface 210. For example, the support structure 220 can be composed of 2, 3, 4, 5, 6, 7, 8, 9, 10, or more or more supporting members 222. The supporting members 222 can form 1, 2, 3, 4, 5, 6, 7, 8, 9, 10 or more cross-shapes on the silicon filtering surface 210. The supporting members 222 can create 2, 3, 4, 5, 6, 7, 8, 9, 10, 11, 12, 13, 14, 15 or more filtering windows 212 in the silicon filtering surface 210.

FIGS. 2A and 2C illustrate example silicon filters 200a and 200c for cell mechanoporation comprising a support structure 220 composed of two supporting members 222 that form a cross-shape, thereby creating four filtering windows 212. FIG. 2E illustrates an example silicon filter comprising a support structure 220 having two cross shapes formed by 3 supporting members 222, thereby creating 6 filtering windows 212. FIGS. 2B, 2D, and 2F illustrate example silicon filters 200b, 200d, and 200f comprising a support structure 220 composed of 4 supporting members 222 that form a plurality of cross-shapes, thereby creating 8 or 9 filtering windows 212. FIG. 2G illustrates an example silicon filter 200g comprising a support structure 220 having 5 supporting members 222 that form a plurality of cross-shapes and 12 filtering windows 212.

The support structure 220 is not limited to the cross-shape(s) described above. For example, as shown in FIGS. 2H, 2I, and 2J, the supporting members 222 of the support structure 220 may create a central filtering window 218 in the silicon filtering surface 210. The supporting member 222 creating the central filtering window 218 may comprise a circular, ovular, triangular, rectangular (e.g., square), or other polygonal shape. In some embodiments, a support structure 220 comprising curved supporting members 222 (e.g., circular supporting members, like central filtering window 218) may improve the filter's ability to withstand pressure and flow conditions induced during cell mechanoporation, at least in comparison to support structures 220 including one or more sharp corners or edges. One or more supporting members 222 may extend from the central supporting member 222 toward the perimeter of the silicon filtering surface 210. For example, 1, 2, 3, 4, 5, 6, 7, 8 or more supporting members 222 may extend from the central supporting member 222, thereby creating one or more filtering windows 218 adjacent to the central filtering window 218. In some examples, the supporting members 222 disposed around the central filtering window 218 may be spaced between about 30° and 180° apart from one another, such as between about 45° and 120° apart, or about 60° and 90° apart. FIG. 2H illustrates an example silicon filter 200h comprising 4 filtering windows 212—1 central window and 3 peripheral windows created by 3 supporting members extending from the central supporting member. The silicon filters 200i and 200j in FIGS. 2I and 2J are similar, but include 5 and 7 filtering windows 218, respectively, created by 5 and 7 supporting members 222 of the support structure 220 (respectively).

In some embodiments, the support structure 220 may comprise one or more supporting members 222 disposed independently as stripes on the silicon filtering surface 210. FIGS. 2K and 2L illustrate example silicon filters 200k and 200l for cell mechanoporation that comprise a plurality of supporting members 222 disposed on the silicon filtering surface 210. Each supporting member 222 of the support structure 220 may extend from a side of the silicon filtering surface 210 toward the side of the silicon filtering surface 210 opposite the aforementioned side. A given supporting member 222 of the support structure 220 may extend substantially all the way from one side to the other (as shown in FIG. 2L) or may extend only part of the way (as shown in FIG. 2K). This concept is described in greater detail herein with respect to the width of the support structure 220, wherein the width of the support structure may be at least 30% of that of the silicon filtering surface 210. The supporting members 222 may be disposed in an alternating fashion, as shown in FIG. 2K, or alternatively may all extend from one side of the silicon filtering surface 210 or the other.

As shown in FIGS. 2A-2L, the silicon filter 200 (e.g., filters 200a-200l, collectively referred to herein as filter 200) for cell mechanoporation may comprise a border surrounding the silicon filtering surface 210. The border may be a non-porous material—in other words, the border may not comprise pores 115 therethrough. The region of the silicon filter 200 bordering the silicon filtering surface 210 and the silicon filter 200 itself may otherwise be collectively referred to herein as the silicon chip. This border on the chip may be used to retain the silicon filter 200 within a filter holder or other appropriate receptacle without obstructing the silicon filtering surface 210. In some embodiments, the silicon chip may comprise a circular, triangular, rectangular, elliptical, or other polygonal shape. In some embodiments, the shape of the chip may mimic that of the filtering surface 210. In some embodiments, as shown in FIGS. 2C-2J, the shape of the chip and the silicon filtering surface 210 may be different (e.g., the chip can comprise rectangular shape, whereas the filtering surface 210 can comprise a circular shape).

The size of the chip (e.g., filter 200a) may be otherwise described with reference to the length 202 and width 204 of the chip, as shown at least in FIG. 2A. The length 202 and/or width 204 of the chip may be between 2-15 mm, 4-14 mm, or 5-12 mm. In some examples, the length 202 and/or width 204 of the chip may be greater than or equal to 2, 4, 5, 6, 8, 10, 12, or 14 mm. In some examples, the length 202 and/or width 204 of the chip may be less than or equal to 4, 5, 6, 8, 10, 12, 14, or 15 mm. In the instance the chip is circular, the diameter of the chip may be the length 202 or width 204 described above. In some examples, the length 202 and width 204 of the chip are the same, i.e., the chip is a square. In some examples, the length 202 and width 204 of the chip are different. The size of the chip is not intended to be limited to the disclosure provided herein. In a few non-limiting examples, the size of the chip (length×width) may be 2×2 mm, 5×5 mm, 10×10 mm, 12×12 mm, 14×14 mm, or any dimensions therebetween.

The silicon filter 100 for cell mechanoporation may comprise one or more silicon materials, such as silicon, silicon oxide (e.g., silica), silicon nitride, and/or silicon carbide. For example, the silicon filtering surface 110 may comprise one or more of the aforementioned silicon materials. Likewise, the support structure 120 may comprise one or more of the aforementioned silicon materials. The silicon filtering surface 110 and the support structure 120 may comprise the same or a different silicon material.

The silicon filter 100 for cell mechanoporation may be fabricated from a silicon wafer that is doped. For example, the silicon wafer used to fabricate the silicon filter 100 may be doped with boron, gallium, or phosphorous.

The silicon filter 100 may be coated. One or more sides of the silicon filter 100 may be coated with a coating. For example, the silicon filter 100 may comprise a coating including one or more materials such as gold, silver, platinum, Teflon, polyvinylpyrrolidone, an adhesive coating, surfactants, one or more proteins, adhesion molecules, antibodies, anticoagulants, factors that modulate cellular function, nucleic acids, lipids, carbohydrates, and/or transmembrane proteins. In some embodiments, the silicon filter 100 may not comprise a coating.

As will be described in greater detail below with respect to the fabrication process of the silicon filters for cell mechanoporation, in some embodiments, the silicon filter 100 may comprise one or more oxide layers. For example, an oxide layer may be disposed on the silicon filtering surface 110 between the silicon filtering surface 110 and the support structure 120. The silicon filter 100 may comprise an oxide layer to ease manufacturing each of the silicon filtering surface 110 and the support structure 120 from a silicon wafer. For example, as described in greater detail below, fabrication of the silicon filter 100 may comprise etching a silicon filtering layer and a silicon support layer of a silicon wafer, the silicon layers separated by an oxide layer. With the oxide layer between the filtering layer and the support layer, each of the silicon filtering surface 110 and the support structure 120 can be reliably etched without etching the other side.

In some embodiments, a system comprising the silicon filter 100 and a cell (or cells, such as a plurality of cells of a cell mixture) may be provided. As mentioned above, the silicon filter 100 may be configured to deform a cell from a cell mixture passed through the silicon filter 100, causing a perturbation in the cell that allows a payload to enter the cell cytosol. The cell may be deformed by the silicon filter 100 for a period of time ranging from about 1 μs to at least about 10 ms, such as about 1 μs, 10 μs, 50 μs, 100 μs, 500 μs, 1 ms, 2 ms, 5 ms, or 10 ms. The perturbation in the cell can be defined as a breach in the cell that allows material from outside the cell to move into the cell (e.g., a hole, tear, cavity, aperture, pore, break, gap, perforation, etc.). The perturbation may be caused by pressure induced by mechanical strain and/or shear forces. The perturbation may be in the cell membrane. The perturbation may be transient. In some embodiments, the perturbation in the cell may persist for a duration between 1 second and 5 minutes. For example, the perturbation may persist for at least 1 s, 10 s, 30 s, 1 min, 2 min, 3 min, 4 min, 5 min, or more. In some embodiments, the perturbation may persist for less than 1 s, 10 s, 30 s, 1 min, 2 min, 3 min, 4 min, or 5 min.

In some embodiments, the silicon filter 100 may be configured to receive a large quantity of cells in a single run. For example, the silicon filter 100 may be configured to receive a cell mixture comprising between about 1.0×10⁸ and 1.0×10¹² cells in a single run. In some embodiments, the silicon filter 100 may be configured to receive a cell mixture comprising between about 1.0×10⁸ and 1.0×10¹¹ cells, 1.0×10⁸ and 1.0×10¹⁰ cells, or 1.0×10⁶ and 1.0×10⁹ cells. In some embodiments, the silicon filter 100 may be configured to receive greater than or equal to about 1.0×10⁸ cells, 5.0×10⁸ cells, 1.0×10⁹ cells, 5.0×10⁹ cells, 1.0×10¹⁰ cells, 5.0×10¹⁰ cells, 1.0×10¹¹ cells, or 5.0×10¹¹ cells. In some embodiments, the silicon filter 100 may be configured to receive less than or equal to about 5.0×10⁸ cells, 1.0×10⁹ cells, 5.0×10⁹ cells, 1.0×10¹⁰ cells, 5.0×10¹⁰ cells, 1.0×10¹¹ cells, 5.0×10¹¹ cells, or 1.0×10¹² cells.

In some embodiments, the cell (or cells) is a cell obtained from, or derived from, an individual. In some embodiments, the individual is a mouse. In some embodiments, the individual is a non-human primate. In some embodiments, the individual is a human. In some embodiments, the individual is a mouse, dog, cat, horse, rat, goat or rabbit.

In some embodiments, the cell (or cells) may be a somatic cell, an immortalized cell (e.g., HeLa cell, HEK cell, etc.), a stem cell, or a derivative thereof. In some embodiments, the cell(s) may be a peripheral blood mononuclear cell (PBMC) or a derivative thereof. In some embodiments, the cell(s) may be an immune cell. In some embodiments, the cell(s) may be a T cell, natural killer (NK) cell, monocyte, B cell, or dendritic cell (or mixture thereof). In some embodiments, the cell(s) may be a stem cell, such as a human stem cell. In some embodiments, the cell(s) may be an induced pluripotent stem cell, a hematopoietic cell, or a mesenchymal cell.

### Cell Mechanoporation Systems Including Silicon Filters

In some embodiments, a cell mechanoporation system comprising one or more silicon cell mechanoporation filters may be provided. FIGS. 3A-3C illustrate example cell mechanoporation systems 330 including a silicon cell mechanoporation filter 300.

As shown in FIGS. 3A-3C, cell mechanoporation system 330 may comprise a filter holder 332 fluidly connectable to a cell mixture source 334, one or more silicon cell mechanoporation filters 300 disposed in the filter holder 332, an output reservoir 336 fluidly connected to the filter holder 332 to collect a perturbed cell mixture, and a pump 338 configured to move the cell mixture through the one or more silicon cell mechanoporation filters 300 and into the output reservoir 336.

The one or more silicon cell mechanoporation filters 300 may comprise any one or more features of silicon filters 100 and 200 described herein with respect to FIGS. 1A-1C and 2A-2L, respectively. For example, the one or more silicon filters 300 may comprise a silicon filtering surface, a plurality of pores extending through the silicon filtering surface and configured to perturb cell membranes as a cell mixture from the cell mixture source 334 passes through the plurality of pores, and a support structure disposed on a side of the silicon filtering surface and covering at least a portion (e.g., about 1%) of the silicon filtering surface.

In some embodiments, the one or more silicon filters 300 may comprise a plurality of silicon filters 300. For example, system 330 may comprise any number of silicon filters 300, such as 1, 2, 3, 4, 5, 6, 7, 8, 9, 10 or more silicon filters. The plurality of silicon filters 300 may be disposed in parallel such that each filter of the plurality of silicon filters 300 may be fluidly connected to cell mixture source 334 to receive a portion of the cell mixture, and to the output reservoir 336 to provide the perturbed cell mixture to the reservoir 336. In this manner, the cell mechanoporation system may be able to process larger volumes of fluid. In some embodiments, the plurality of silicon filters 300 may be fluidly connected in series.

The filter holder 332 may comprise a housing, or cartridge, configured to hold the one or more silicon filters 300 (hereinafter referred to as silicon filter 300 for simplicity). The filter holder 332 may comprise a fluid inlet configured to fluidly connect to the cell mixture source 334. The filter holder 332 may comprise a fluid outlet configured to fluidly connect to the output reservoir 336. The filter holder 332 may be removably connectable to the cell mixture source 334 and/or the output reservoir 336. In some embodiments, the filter holder 332 may comprise the output reservoir 336. In some embodiments, the filter holder 332 may be replaceable within the cell mechanoporation system 330. In some embodiments, the silicon filter 300 may be replaceable within the filter holder 332.

In some embodiments, system 330 may comprise one or more leukocyte reduction filters and/or cell strainers (not illustrated). For example, filter holder 332 may hold one or more leukocyte reduction filters upstream of silicon filter 300, the leukocyte reduction filters configured to remove white blood cells from the cell mixture prior to the cell mixture being passed through the silicon filter 300. Likewise, cell strainers may be integrated within system 330 (e.g., held by filter holder 332 upstream of silicon filter 300) and configured to remove larger clumps (e.g., clumps within a size range of 40-70 μm) that may otherwise clog the silicon filter 300. The one or more leukocyte reduction filters and/or cell strainers may be fluidly connected to cell mixture source 334 to receive the cell mixture (e.g., the cell suspension and optionally the payload). In some embodiments, the system 330 may not comprise leukocyte reduction filter(s). In some embodiments, the system 330 may not comprise cell strainer(s). For example, one or more of the leukocyte reduction filters and/or cell strainers may instead be integrated within a fluid delivery system upstream of the silicon filter 300.

Cell mixture source 334 may comprise a cell mixture (e.g., a cell suspension). The cell mixture may comprise cells and a buffer solution. For example, the cell mixture may comprise a mixture of cells in a physiological saline solution or physiological medium other than blood. The cells in the cell mixture may include but are not limited to a mixed cell population (e.g., whole blood) or a purified cell population (e.g., a purified nucleate or anucleate cell population). As described above, the cells in the cell mixture may include but are not limited to anucleate cells, (e.g., red blood cells such as erythrocytes or reticulocytes, platelets, etc.), nucleate cells (e.g., immune cells, PBMCs, T cells, NK cells, iPSCs, HSCs, etc.), liposomes, exosomes, etc. The cell mixture may comprise an aqueous solution including a cell culture medium (e.g., DMEM, RMPI, OptiMEM, etc.), phosphate-buffered saline (PBS), salts, sugars, growth factors, surfactants, lubricants, amino acids, proteins, cell cycle inhibitors, vitamins, etc.

As described herein, the silicon filter 300 provided herein may be integrated with clinical-scale cell processing systems. These systems may provide several million, hundred million, billion, or even trillion cells to the filter for cell mechanoporation. Thus, the silicon filters described herein may be configured to receive large quantities of cells (e.g., billions of cells) and may deliver a payload to these cells with high efficacy. Given that flow through a porous surface is not as controllable as, for example, microfluidic channels, the ability to deliver a payload to millions of cells in a cell mixture with high efficacy using the silicon filters 300 described herein may be important for successfully integrating the silicon filters described herein in cell processing systems of various magnitudes. Moreover, a single silicon cell mechanoporation filter 300 described herein may be configured to process millions (or billions) of cells in a single run without replacing the filter.

In some embodiments, the cell mixture may comprise between about 1.0×10⁸ and 1.0×10¹² cells. In some embodiments, the cell mixture may comprise between about 1.0×10⁸ and 1.0×10¹¹ cells, 1.0×10⁸ and 1.0×10¹⁰ cells, or 1.0×10⁶ and 1.0×10⁹ cells. In some embodiments, the cell mixture may comprise greater than or equal to about 1.0×10⁸ cells, 5.0×10⁸ cells, 1.0×10⁹ cells, 5.0×10⁹ cells, 1.0×10¹⁰ cells, 5.0×10¹⁰ cells, 1.0×10¹¹ cells, or 5.0×10¹¹ cells. In some embodiments, the cell mixture may comprise less than or equal to about 5.0×10⁸ cells, 1.0×10⁹ cells, 5.0×10⁹ cells, 1.0×10¹⁰ cells, 5.0×10¹⁰ cells, 1.0×10¹¹ cells, 5.0×10¹¹ cells, or 1.0×10¹² cells.

In some embodiments, the cell mixture has a volume of about 10 μL to about 5 L, such as at least about 10 μL, 100 μL, 500 μL, 1 mL, 5 mL, 10 mL, 50 mL, 100 mL, 200 mL, 300 mL, 400 mL, 500 mL, 600 mL, 700 mL, 800 mL, 900 mL, 1 L, 2 L, 3 L, 4 L, or 5 L. In some embodiments, the cell mixture may have a volume between about 10-1000 μL, 10-500 μL, 10-100 μL, 10-50 μL, 50-1000 μL, 50-500 μL, 50-100 μL, 100-1000 μL, 100-500 μL, 500-1000 μL, 1-1000 mL, 1-500 mL, 1-100 mL, 1-50 mL, 1-10 mL, 10-1000 mL, 10-500 mL, 10-100 mL, 10-50 mL, 50-1000 mL, 50-500 mL, 50-400 mL, 50-300 mL, 50-200 mL, 50-100 mL, 100-1000 mL, 100-500 mL, 100-400 mL, 100-300 mL, 100-200 mL, 500-1000 mL, or 1-5 L. As mentioned above, the silicon filters described herein (and therefore the cell mechanoporation system comprising the silicon filter(s)) may be configured to process a large range of fluid quantities, dependent on whether the silicon filter is integrated with a research-scale system, clinical-scale system, high-throughput screening system, etc.

Cell mixture source 334 may comprise a fluid reservoir, flexible plastic bag, vial, bottle, vessel, ampule, jar, or other container suitable for containing the cell mixture. In another example, cell mixture source 334 may not comprise a separate vessel containing the cell mixture, but rather the system 330 may be configured to receive the cell mixture from an upstream, fluidly connectable cell therapy production system, cell washing system, filtration system, etc. For example, one or more of the aforementioned systems may process the cells prior to providing the mixture to system 330 by surface marker-based separation (e.g. Miltenyi), centrifugation/flow-based separation (e.g. elutriation), buffer exchanges, cell washing, activations, expansions, or other biological variations.

Example filtration and/or elutriation systems that may be fluidly connectable to filter holder 332 and comprise cell mixture source 334 may include but are not limited to tangential flow filtration (TFF) systems, (e.g., Repligen KrosFlo® KR2i TFF system, Sartorius Ambr® Crossflow TFF system, Sartorius Sartoflow® Smart TFF system, etc.), standard filtration systems (e.g., Fresenius Cue® Cell Processing System), spinning membrane filtration, (e.g., Fresenius Kabi Lovo® Automated Cell Processing System), and elutriation systems (e.g., Gibco™ Cell Therapy Systems (CTS™) Rotea Counterflow Centrifugation System, Terumo Elutra® Cell Separation System, etc.). Example cell isolation systems include but are not limited to the Miltenyi CliniMACS Prodigy® instrument and StemCell cell isolation systems (e.g., immunomagnetic cell separation systems such as EasySep™, RoboSep™ and StemSep™ immunodensity cell separation systems such as RosetteSep™ and SepMate™, etc.). Example end-to-end cell therapy production systems include but are not limited to the Lonza Cocoon® Platform.

In some embodiments, silicon filter 300 (e.g., disposed in a filter holder 332) may be integrated into (e.g., as a component of) one or more of the aforementioned pumps, filtration systems, isolation systems, cell therapy production systems, and/or elutriation systems.

In some embodiments, the cell mixture may comprise the cell suspension and a payload (e.g., a pre-mixed cell suspension and payload). The type of payload as well as the concentration thereof may be dependent on the type of cells in the cell suspension and the intended cell therapy. In some embodiments, the payload comprises at least one of a polypeptide, a nucleic acid, a small molecule, a nanoparticle, and a complex thereof. For example, the payload may include but is not limited to one or more of DNA, RNA (e.g., mRNA, siRNA, saRNA, tRNA, miRNA, etc.), proteins, small molecules, peptides, nanoparticles, viruses, synthetic materials, a cell lysate comprising an antigen, and/or complexes (e.g., RNP, protein CAS plus guide RNA, etc.). The payload may comprise a plurality of payloads (e.g., a mixture of nucleic acids, proteins, small molecules, etc.) The aforementioned payloads may function biologically as transcription factors, chemokines, cytokines, surface receptors, other intracellular or extracellular proteins, inhibitors or enhancers of any of the above, survival factors, cryoprotectants, prime editors, antibodies, enzymes, and/or any combinations of the above.

In some embodiments, the payload may comprise at least one molecule having a molecular weight between about 100 Da and 10 MDa. For example, as described herein, the payload may comprise small molecules (˜100 Da) and/or large plasmids (˜10 MDa). For example, a molecular weight of a molecule in the payload may be between about 100 Da and 1 MDa, 100 Da and 100 kDa, or 100 Da and 1 kDa. In some embodiments, the payload may comprise at least one molecule having a molecular weight greater than or equal to about 100 Da, 500 Da, 1 kDa, 50 kDa, 100 kDa, 500 kDa, 1 MDa, or 5 MDa. In some embodiments, the payload may comprise at least one molecule having a molecular weight less than or equal to about 500 Da, 1 kDa, 50 kDa, 100 kDa, 500 kDa, 1 MDa, 5 MDa, or 10 MDa. As described herein, the payload may comprise a plurality of molecules, particles, etc. having different molecular weights selected from those listed above.

As the mechanoporation mechanism described herein is dependent on the payload transfecting into a pore of the cell membrane, the diameter of the payload (e.g., particle, molecule, etc.) may be less than 100 nm. For example, the particle size may be between about 1-100 nm, 1-50 nm, or 1-10 nm. In some embodiments, the particle size (diameter) may be greater than or equal to 1, 5, 10, 25, 50, or 75 nm. In some embodiments, the particle size (diameter) may be less than or equal to 5, 10, 25, 50, 75, or 100 nm.

The concentration of the payload in the cell mixture may vary based on the type of payload and may be between about 1 nM and 1 mM. For example, the payload concentration may be between about 10 nM-1 mM, 100 nM-1 mM, 1 μm-1 mM, 10 μM-1 mM, or 100 μM-1 mM. In some embodiments, the concentration of the payload in the cell mixture may be greater than or equal to 1 nM, 10 nM, 50 nM, 100 nM, 500 nM, 1 μm, 5 μm, 10 μm, 50 μm, 100 μm, 500 μm, or 1 mM. In some embodiments, the concentration of the payload in the cell mixture may be less than or equal to 1 nM, 10 nM, 50 nM, 100 nM, 500 nM, 1 μm, 5 μm, 10 μm, 50 μm, 100 μm, 500 μm, or 1 mM.

In some embodiments, the payload may be contained in a payload source 335. As shown in FIG. 3B, the payload source 335 may be fluidly connectable to the cell mixture source 334 to receive the cell mixture prior to passing the cell mixture and payload together to the silicon cell mechanoporation filter 300. As shown in FIG. 3C, the payload source 335 may additionally or alternatively be fluidly connectable to the output reservoir 336 to provide the payload to the output reservoir 336 (also configured to receive the perturbed cell mixture from the silicon cell mechanoporation filter 300). In this example, the payload may be introduced to the perturbed cells of the cell suspension after the cell suspension is passed through the silicon filter 300. Payload source 335 may comprise a fluid reservoir, flexible plastic bag, vial, bottle, vessel, ampule, jar, or other container suitable for containing the payload.

In some embodiments, the payload may be contained in the output reservoir 336 fluidly connected to the silicon filter 300 (e.g., understood to be illustrated by system 330 in FIG. 3A). That is, mechanoporation system 330 may not comprise an external payload source 335. In each of these aforementioned embodiments, it is contemplated that the payload (e.g., included in cell mixture source 334, output reservoir 336, or payload source 335) may be combined with the cell mixture before, during, and/or after the cell mixture is passed through the silicon filter 300.

One or more of the payload and/or cell mixture may comprise a solution intended to prime fluid pathways and/or reservoirs of system 330. For example, the payload may comprise a cell buffer and/or payload buffer. In some embodiments, cell mechanoporation system 330 may be used without priming (or wetting). For example, fluid reservoirs and/or pathways of system 330 may be pre-wetted.

Cell mechanoporation system 330 may comprise one or more pumps 338 configured to move the cell mixture through the one or more silicon filters 300 and into the output reservoir 336. The one or more pumps 338 (hereinafter referred to as pump 338 for simplicity) may be configured to provide positive pressure or negative pressure (e.g., a vacuum) to move the cell mixture (i.e., the cell suspension and/or payload) through system 330. For example, negative pressure from pump 338 may “pull” one or more of the aforementioned solutions through the system 330, whereas positive pressure may “push” or drive the solutions through the system 330. The one or more pumps 338 may comprise a single pump 338.

In some embodiments, the pump 338 may comprise a peristaltic pump or a syringe pump. Pump 338 may include but is not limited to peristaltic pumps, syringe pumps, diaphragm pumps, positive displacement pumps, gear lobes, etc. More generally, the term “pump” may further be used herein to refer to manual syringes, pipettes, pistons, gas cylinders, and/or other fluid motivators (e.g., gravity, centripetal force, etc.). In some embodiments, the pump 338 may be a pump of an existing filtration system, isolation system, elutriation system, cell washing system, and/or end-to-end cell therapy production system (described above).

As illustrated in FIG. 3A, the pump 338 may be operably coupled to at least one of the filter holder 332, cell mixture source 334, and/or output reservoir 336 (i.e., one or more fluid pathways fluidly connecting the filter holder 332 to each of the cell mixture source 334 and output reservoir 336) to move the cell mixture from the cell mixture source 334, through the silicon filter 300, and into the output reservoir 336. As illustrated in FIGS. 3B and 3C, in the instance system 330 comprises a payload source 335, the pump 338 may additionally or alternatively be coupled to the payload source 335 (i.e., one or more fluid pathways fluidly connected to the payload source 335).

The pump 338 may be configured to provide a pressure (e.g., a pulsed pressure, a constant pressure, etc.) to the cell mechanoporation system 330 between at least 2 psi and 35 psi. For example, the pump 338 may provide a pressure between at least 2-20 psi, 2-10 psi, 10-35 psi, or 10-20 psi. In some embodiments, the pressure provided by pump 338 may be greater than or equal to about 1 psi, 5 psi, 10 psi, 15 psi, 20 psi, 25 psi, 30 psi, or 35 psi. In some embodiments, the pressure provided by pump 338 may be less than or equal to about 1 psi, 5 psi, 10 psi, 15 psi, 20 psi, 25 psi, 30 psi, or 35 psi. In some embodiments, system 330 may comprise one or more components (e.g., one or more valves, pressure dampeners, etc.) configured to control or modify (e.g., step-down) the pressure provided by the pump 338. In doing so, a pressure gradient across the one or more silicon filter 300 may be maintained within the intended range (e.g., between 2 μsi and 20 psi). In some examples, the pressure gradient across the silicon filtering surface may be less than 20 psi, such as 18 psi, 15 psi, 14 psi, 12 psi, 10 psi, 8 psi, 5 psi, or psi. In some examples, the pressure gradient across the silicon filtering surface may be between 2-20 psi, 5-20 μsi, 8-20 psi, or 10-20 psi. In some examples, the pressure gradient across the silicon filtering surface may be less than 2 psi, such as 1.5 psi, 1 psi, 0.5 psi, or 0.25 psi. In some embodiments, the pressure gradient across the silicon filtering surface may be greater than 15 psi, such as 17.5 μsi, 20 psi, 22.5 psi, 25 psi, 30 psi, or more. The silicon filters described herein can be configured such that at the above-described low-pressure gradients, and using the flow rates described herein, the filtering surface can effectively perturb cell membranes for payload delivery.

The pump 338 may be configured to pump (e.g., move) a total volume between at least 1 mL and 500 mL through the system 330 (e.g., through the silicon filter 300) in a single run. For example, pump 338 may be configured to pump between about 1-100 mL, 1-50 mL, 10-500 mL, 10-100 mL, or 100-500 mL through system 330 in a single run. In some embodiments, pump 338 may be configured to drive greater than or equal to about 1 mL, 50 mL, 100 mL, 200 mL, 300 mL, 400 mL, or 500 mL of fluid through the system 330. In some embodiments, pump 338 may be configured to drive less than or equal to about 1 mL, 50 mL, 100 mL, 200 mL, 300 mL, 400 mL, or 500 mL of fluid through the system 330. In some embodiments, the pump 338 may be configured to pump less than 1 mL of fluid through the system in a single run, such as a minimum volume of 100 μL, 250 μL, 500 μL, or 750 μL. In some embodiments, the pump 338 may be configured to pump greater than 500 mL of fluid through the system in a single run, such as about 1 L, 2 L, 3 L, 4 L, or 5 L. In some embodiments, the system 330 as described herein may be adapted for allogeneic production and/or bioprocessing, and thus the pump 338 may be configured to pump a volume greater than 5 L, such as 10 L, 100 L, or 1000 L.

In some embodiments, the cell mechanoporation system 330 may be operable at a constant volumetric flow rate. In other words, the pump 338 of the system 330 may be configured to provide a constant volumetric flow rate to the silicon filter 300. In some embodiments, the pump 338 may be configured to provide a pulsed volumetric flow rate to the silicon filter 300. In some embodiments, in addition to or instead of the pump 338, the volumetric flow (pulsed or constant) may be controlled by a pressurized reservoir.

As described herein, the silicon filter 300 may be configured to receive a volumetric flow rate between 0.5-500 mL/min per mm² porous surface area. In other words, the pump 338 may be configured to move the cell mixture through the silicon filter 300 at the volumetric flow rate between about 0.5-500 mL/min per mm² porous surface area. In some embodiments, the pump 338 may be configured to pump the cell mixture through the silicon filter 300 at a volumetric flow rate greater than or equal to about 0.5, 1, 2, 2.5, 3, 4, 5, 6, 7, 8, 9, 10, 12, 15, 18, 20, 25, 30, 35, 40, 45, 50, 75, 100, 125, 150, 175, 200, 250, 300, 350, 400, or 450 mL/min per mm² porous surface area. In some embodiments, the pump 338 may be configured to pump the cell mixture through the silicon filter 300 at a volumetric flow rate less than or equal to about 1, 2, 2.5, 3, 4, 5, 6, 7, 8, 9, 10, 12, 15, 18, 20, 25, 30, 35, 40, 45, 50, 75, 100, 125, 150, 175, 200, 250, 300, 350, 400, 450, or 500 mL/min per mm² porous surface area.

In some embodiments, all of the cell mixture may be passed through the silicon filter 300 in about 30 minutes or less. In some embodiments, all of the cell mixture may be passed through the silicon filter 300 in no more than about 30 minutes, 25 minutes, 20 minutes, 15 minutes, 12 minutes, 10 minutes, 8 minutes, 5 minutes, 2 minutes, or 1 minutes. In some embodiments, all of the cell mixture may be passed through the silicon filter 300 in about 1-30 minutes, 1-25 minutes, 1-20 minutes, 1-15 minutes, 1-10 minutes, 1-5 minutes, 5-30 minutes, 5-25 minutes, 5-20 minutes, 5-15 minutes, 5-10 minutes, 10-30 minutes, 10-25 minutes, 10-20 minutes, or 10-15 minutes. The time in which the cell mixture can be passed through the silicon filter 300 may be at least partially driven by the pump 338 and/or upstream cell processing system(s).

In some embodiments, a single cell of the cell mixture may be passed through the silicon filter 300 for about 1 microsecond to 10 milliseconds. For example, a cell may be passed through the silicon filter 300 within 1 μs-1 ms, 10 μs-10 ms, 10 μs-1 ms, 100 μs-10 ms, or 100 μs-1 ms. In some embodiments, a cell may be passed through the silicon filter 300 in greater than or equal to about 1 μs, 5 μs, 10 μs, 50 μs, 100 μs, 500 μs, 1 ms, or 5 ms. In some embodiments, a cell may be passed through the silicon filter 300 in less than or equal to about 5 μs, 10 μs, 50 μs, 100 μs, 500 μs, 1 ms, 5 ms, or 10 ms.

In some embodiments, the speed of the cells through the silicon filter 300 to induce cell membrane perturbation may be about 1 m/s. In some embodiments, the speed of the cells may be between 10 mm/s and 100 m/s. For example, the speed of the cells may be less than or equal to 10 mm/s, 20 mm/s, 50 mm/s, 1 m/s, 2 m/s, 5 m/s, 10 m/s, 20 m/s, 50 m/s, or 100 m/s. In some embodiments, the speed of the cells may be greater than or equal to 10 mm/s, 20 mm/s, 50 mm/s, 1 m/s, 2 m/s, 5 m/s, 10 m/s, 20 m/s, 50 m/s, or 100 m/s.

Mechanoporation system 330 may comprise one or more output reservoirs 336 fluidly connected to the silicon filter 300 to collect the perturbed cell mixture. The one or more output reservoirs 336 may comprise a single output reservoir 336. Alternatively, the one or more output reservoirs 336 may comprise a plurality of output reservoirs 336 (hereinafter referred to as output reservoir 336). As described herein, the aforementioned pump 338 may be configured to move the perturbed cell mixture into the output reservoir 336. Output reservoir 336 may comprise one or more flexible plastic bags, vials, vessels, or other containers configured to collect the perturbed cell mixture. As described herein, it is contemplated that the output reservoir 336 may be contained within a housing comprising the filter holder 332.

In some examples, the sample volume may be such that a residence time of the cells in the output reservoir is minimized. Residence time is measured from the time the cell exits the filter to when it leaves the output reservoir (e.g., in system 330 depicted in FIG. 3A) or is exposed to a payload (e.g., in system 330 depicted in FIG. 3C). For example, the residence time of the cells in the output reservoir may be less than 30 seconds (s), less than 20 s, less than 15 s, less than 10 s, or less than 5 s. In some examples, the residence time is between about 0.01 seconds to about 30 seconds. Other features of the system and parameters for operation thereof may contribute to residence time, such as flow rate and cell concentration. From description of such variables elsewhere in the instant application, one of ordinary skill in the art will readily appreciate their ability to affect the residence time.

The perturbed cell mixture produced from passing the mixture through the silicon filter 300 may be incubated (e.g., the cells may be frozen) for a duration of time. For example, the perturbed cell mixture may be incubated for about 0.0001 s to 20 min, such as 1 s, 30 s, 90 s, 2 min, 5 min, 10 min, 15 min, or 20 min. The perturbed cell mixture may be incubated at a temperature between 0° C. and 40° C., such as at least 0° C., 5° C., 10° C., 20° C., 25° C., 30° C., 35° C., or 40° C. The perturbed cell mixture may be incubated in output reservoir 336. In some embodiments, the perturbed cell mixture may instead or additionally be washed prior to incubation. Finally, in combination with one or more of the aforementioned post-processing steps and/or immediately following collection, the perturbed cell mixture may be injected to a patient to execute the intended cell therapy with the cells.

### Methods of Intracellular Delivery of a Payload Using Silicon Cell Mechanoporation Filters

In some embodiments, a method for intracellular delivery of a payload may be provided. The method may comprise passing a cell mixture comprising cells and a payload through a plurality of pores extending through a silicon filtering surface of a silicon cell mechanoporation filter. The silicon cell mechanoporation filter may comprise any one or more features of silicon cell mechanoporation filters 100, 200, and 300 described herein with respect to FIGS. 1A-1C, 2A-2J, and 3A-3C, respectively. For example, the silicon cell mechanoporation filter may comprise a support structure disposed on a side of the silicon filtering surface and covering at least a portion (e.g., 1%) of the silicon filtering surface. The method may comprise collecting a perturbed cell mixture comprising the cells with the payload in the cells.

In some embodiments, the cell mixture passed through the plurality of pores in accordance with the methods described herein may have a volume between at least 10 μL and 1000 L. For example, the cell mixture may have a volume between about 1-1000 μL, 1-100 μL, 1-1000 mL, 1-100 mL, or 1-1000 L. In some embodiments, the cell mixture may have a volume greater than or equal to about 10 μL, 50 μL, 100 μL, 500 μL, 1 mL, 5 mL, 10 mL, 20 mL, 50 mL, 100 mL, 500 mL, 1 L, 2 L, 5 L, 10 L, 50 L, 100 L, or 500 L. In some embodiments, the cell mixture may have a volume less than or equal to about 50 μL, 100 μL, 500 μL, 1 mL, 5 mL, 10 mL, 20 mL, 50 mL, 100 mL, 500 mL, 1 L, 2 L, 5 L, 10 L, 50 L, 100 L, 500 L, or 1000 L.

In some embodiments, the cell mixture may be passed through the plurality of pores at a constant volumetric flow rate. In some embodiments, the cell mixture may be passed through the plurality of pores in accordance with a pulsed volumetric flow rate. For example, the flow of the cell mixture through the plurality of pores may be controlled by at least one of a peristaltic pump, a syringe pump, and a pressurized reservoir.

In some embodiments, the cell mixture may be passed through the plurality of pores at a volumetric flow rate between about 0.5-500 mL/min per mm² porous surface area. For example, the cell mixture may be passed through the plurality of pores at a volumetric flow rate of about 0.5-100, 0.5-10, 50-500, 5-100, or 5-20 mL/min per mm² porous surface area. In some embodiments, the cell mixture may be passed through the plurality of pores at a volumetric flow rate greater than or equal to about 0.5, 1, 2, 5, 10, 15, 20, 30, 40, 50, 75, 100, 125, 150, 175, 200, 250, 300, 350, 400, or 450 mL/min per mm² porous surface area. In some embodiments, the cell mixture may be passed through the plurality of pores at a volumetric flow rate less than or equal to about 1, 2, 5, 10, 15, 20, 30, 40, 50, 75, 100, 125, 150, 175, 200, 250, 300, 350, 400, 450, or 500 mL/min per mm² porous surface area.

In some embodiments, all of the cell mixture may be passed through the plurality of pores in about 30 minutes or less. In some embodiments, all of the cell mixture may be passed through the plurality of pores in no more than about 30 minutes, 25 minutes, 20 minutes, 15 minutes, 12 minutes, 10 minutes, 8 minutes, 5 minutes, 2 minutes, or 1 minutes. In some embodiments, all of the cell mixture may be passed through the silicon filter 300 in about 1-30 minutes, 1-25 minutes, 1-20 minutes, 1-15 minutes, 1-10 minutes, 1-5 minutes, 5-30 minutes, 5-25 minutes, 5-20 minutes, 5-15 minutes, 5-10 minutes, 10-30 minutes, 10-25 minutes, 10-20 minutes, or 10-15 minutes.

In some embodiments, a single cell of the cell mixture may be passed through a pore of the plurality of pores for about 1 microsecond to 10 milliseconds. For example, a cell may be passed through a pore within 1 μs-1 ms, 10 μs-10 ms, 10 μs-1 ms, 100 μs-10 ms, or 100 μs-1 ms. In some embodiments, a cell may be passed through a pore in greater than or equal to about 1 μs, 5 μs, 10 μs, 50 μs, 100 μs, 500 μs, 1 ms, or 5 ms. In some embodiments, a cell may be passed through a pore in less than or equal to about 5 μs, 10 μs, 50 μs, 100 μs, 500 μs, 1 ms, 5 ms, or 10 ms.

In some embodiments, the speed of the cells through the plurality of pores to induce cell membrane perturbation may be about 1 m/s. In some embodiments, the speed of the cells may be between 10 mm/s and 100 m/s. For example, the speed of the cells may be less than or equal to 10 mm/s, 20 mm/s, 50 mm/s, 1 m/s, 2 m/s, 5 m/s, 10 m/s, 20 m/s, 50 m/s, or 100 m/s. In some embodiments, the speed of the cells may be greater than or equal to 10 mm/s, 20 mm/s, 50 mm/s, 1 m/s, 2 m/s, 5 m/s, 10 m/s, 20 m/s, 50 m/s, or 100 m/s.

In some embodiments, the method for intracellular delivery of the payload may comprise, prior to passing the cell mixture through the plurality of pores in the silicon filtering layer, priming the silicon filter. For example, the silicon filter may be primed with a cell buffer (e.g., cell media). In some embodiments, the cell mixture may comprise the cell mixture for priming. In some embodiments, the silicon filter may be pre-wetted. In some embodiments, the silicon filter may not require priming.

In some examples, the sample volume may be such that a residence time of the cells in the output reservoir is minimized. For example, the residence time of the cells in the output reservoir may be less than 30 seconds (s), less than 20 s, less than 15 s, less than 10 s, or less than 5 s. In some examples, the residence time is between about 0.01 seconds to about 30 seconds.

As described herein, the cell mixture passed through the plurality of pores may comprise a somatic cell, an immortalized cell (e.g., HeLa cell, HEK cell, etc.), a stem cell, or a derivative thereof. In some embodiments, the cell mixture comprises a PBMC or a derivative thereof. In some embodiments, the cell mixture comprises an immune cell. In some embodiments, the cell mixture comprises a T cell, NK cell, monocyte, B cell, or dendritic cell. In some embodiments, the cell mixture comprises a stem cell, such as a human stem cell. In some embodiments, the cell mixture comprises an induced pluripotent stem cell, a hematopoietic cell, or a mesenchymal cell. In some embodiments, the cell mixture comprises a cell obtained or derived from an individual, such as a human.

In some embodiments, the method for intracellular delivery of the payload may be utilized for intracellular delivery of payload to a large quantity of cells, such as at least about 100 million cells. For example, the cell mixture may comprise a number of cells on the magnitude of millions, hundreds of millions, billions, or even a trillion cells. In some embodiments, the method may comprise passing a cell mixture comprising at least 1.0×10⁸ cells, such as between about 1.0×10⁸ and 1.0×10¹² cells through the plurality of pores. In some embodiments, at least about 5.0×10⁸ cells, 1.0×10⁹ cells, 5.0×10⁹ cells, 1.0×10¹⁰ cells, 5.0×10¹⁰ cells, 1.0×10¹¹ cells, or 5.0×10¹¹ cells may be passed through the plurality of pores. In some embodiments, this large quantity of cells can be passed through the filter in a single run without replacing the filter. In some embodiments, only a single silicon cell mechanoporation filter is needed to efficaciously facilitate intracellular delivery in the several hundred million cells in the cell mixture.

In some embodiments, the cell mixture comprises a payload, such that perturbing the cells in the cell mixture facilitates entry of the payload into the cell (i.e., the cell cytosol). In some embodiments, the method may comprise mixing the cell mixture (i.e., a cell suspension) with a payload prior to passing the cell mixture through the plurality of pores in the silicon filtering layer. In some embodiments, the cell mixture may comprise a pre-mixed payload and cell suspension. In some embodiments, the method comprises introducing a payload to the cell mixture after cell perturbation, thereby facilitating entry of the payload into the cell. The payload may comprise at least one of a polypeptide, nucleic acid, a small molecule, a nanoparticle, and a complex thereof. For example, the payload may comprise a mixture of DNA, RNA, small molecules, nanoparticles, etc.

In some embodiments, the aforementioned method may be performed at a temperature between about 0° C. and about 40° C. For example, the method may be carried out at room temperature (e.g., about 20° C.), physiological temperature (e.g., about 37° C.), higher than physiological temperature (e.g., about 37° C. or more), or at a reduced temperature (e.g., about 0° C. to 4° C.).

### Methods of Fabricating Silicon Cell Mechanoporation Filters

In some embodiments, a method of fabricating (i.e., manufacturing) a silicon filter for cell mechanoporation is provided herein. FIGS. 4A-4F illustrate an example method for fabricating silicon cell mechanoporation filters. The Figures illustrate a single silicon cell mechanoporation filter, however, it is to be understood that the method is intended to capture fabrication of one or more (e.g., a plurality of) silicon cell mechanoporation filters.

Manufacturing a silicon cell mechanoporation filter may begin with a silicon wafer 450. As shown in FIG. 4A, the silicon wafer 450 may comprise a layered structure. For example, the silicon wafer 450 may comprise a first silicon layer 452 (hereinafter referred to as filtering layer 452), a second silicon layer 454 (hereinafter referred to as support layer 454), and an oxide layer 456 disposed between the filtering layer 452 and the support layer 454. In other words, the oxide layer 456 may be on a side of the filtering layer 452, and the support layer 454 may be on a side of the oxide layer 456 opposite the filtering layer 452.

In some embodiments, a thickness of the filtering layer 452 before fabrication of the silicon filter may be about 1-50 μm. For example, the initial thickness of the filtering layer 452 may be between about 1-25 μm, 1-10 μm, 10-50 μm, 10-25 μm, or 25-50 μm. In some embodiments, the thickness of the filtering layer 452 prior to fabrication of the silicon filter may be less than or equal to about 50 μm, 45 μm 40 μm 35 μm, 30 μm, 25 μm, 20 μm, 18 μm, 15 μm, 12 μm, 10 μm, 8 μm, 5 μm, or 2 μm. In some embodiments, the thickness of the filtering layer 452 prior to fabrication may be greater than or equal to about 1 μm, 2 μm, 5 μm, 8 μm, 10 μm, 12 μm, 15 μm, 18 μm, 20 μm, 25 μm, 30 μm, 35 μm, 40 μm, or 45 μm.

In some embodiments, a thickness of the support layer 454 before fabrication of the silicon filter may be between about 100-500 μm. For example, the initial thickness of the support layer 454 may be between about 100-400 μm, 100-200 μm, 200-500 μm, or 200-400 μm. In some embodiments, the thickness of the support layer 454 prior to fabrication of the silicon filter may be less than or equal to 500 μm, 450 μm, 400 μm, 350 μm 300 μm, 250 μm, 200 μm, or 150 μm. In some embodiments, the thickness of the support layer 454 prior to fabrication of the silicon filter may be greater than or equal to 100 μm, 150 μm, 200 μm, 250 μm, 300 μm, 350 μm, 400 μm, or 450 μm.

In some embodiments, a thickness of the oxide layer 456 may be between about 0.1-10 μm. For example, the oxide layer 456 thickness may be between about 0.1-5 μm, 0.1-1 μm, 1-10 μm, 1-5 μm, or 5-10 μm. In some embodiments, the thickness of the oxide layer 456 may be greater than or equal to about 0.1 μm, 0.2 μm, 0.5 μm, 0.8 μm, 1 μm, 1.2 μm, 1.5 μm, 1.8 μm, 2 μm, 2.5 μm, 3 μm, 3.5 μm, 4 μm, 4.5 μm, 5 μm, 6 μm, 7 μm, 8 μm, or 9 μm. In some embodiments, the thickness of the oxide layer 456 may be less than or equal to about 10 μm, 9 μm, 8 μm, 7 μm, 6 μm, 5 μm, 4.5 μm, 4 μm, 3.5 μm, 3 μm, 2.5 μm, 2 μm, 1.8 μm, 1.5 μm, 1.2 μm, 1 μm, 0.8 μm, 0.5 μm, or 0.2 μm.

In some embodiments, the silicon wafer 450 may comprise a second oxide layer (not illustrated) on a side of the support layer 454 opposite the oxide layer 456 (i.e., the first oxide layer). The thickness of the second oxide layer may be understood to be a thickness described above with regards to oxide layer 456. For example, a thickness of the second oxide layer may be between about 0.1-10 μm.

In some embodiments, the silicon wafer 450 may comprise silicon, such as silicon, silicon oxide, silicon nitride, silicon carbide, etc.

In some embodiments, the silicon wafer 450 may be doped. For example, the method may comprise doping the silicon wafer 450, or the silicon wafer 450 may be provided doped. The silicon wafer 450 may be doped with boron, gallium, phosphorous, etc.

In some embodiments, the silicon wafer 450 may be coated. For example, the method may comprise coating the silicon wafer and/or the silicon wafer may be provided coated. Coating the silicon wafer 450 may comprise using low-pressure chemical vapor deposition (e.g., at a desired temperature, such as about 850° C.). The silicon wafer 450 may be coated with gold, silver, or platinum. In some embodiments, the silicon wafer 450 may be provided coated (e.g., with gold, silver, platinum, etc.), and the method may comprise coating the silicon cell mechanoporation filter 400 with another coating (e.g., an adhesive coating, surfactant, anticoagulant, etc.). The method may comprise coating the silicon filter 400 after a fabrication step described herein (e.g., following cleaning the wafer 450 after etching the pores 415 and/or the support structure 420). In some embodiments, one or both sides of the silicon filter 400 may be coated (e.g., with the same or a different coating). In some embodiments, the silicon wafer 450 and/or the silicon filter 400 may not be coated.

As shown in FIG. 4B, the method may comprise creating a plurality of pores 415 in the filtering layer 452 of the silicon wafer 450. In some embodiments, creating the plurality of pores 415 may comprise applying a photoresist film layer comprising a pore template to the filtering layer 452 and etching the plurality of pores 415 in accordance with the pore template. For example, an AZ1512 positive photoresist film layer may be applied to the filtering layer 452 with an exposure dose of 180 mJ. In some embodiments, the photoresist film layer may comprise a thickness between 0.1-5 μm, such as about 1 μm, 1.2 μm, 1.5 μm, 1.7 μm, or 2 μm. In some embodiments, etching the plurality of pores 415 may comprise deep reactive ion etching the plurality of pores 415 linearly through the filtering layer 452 and to the oxide layer 456, as shown in FIG. 4B. FIG. 4C illustrates a top view of the pores 415 in the silicon wafer 450.

In some embodiments, the plurality of pores 415 etched to the filtering layer 452 may have a width between about 2 μm and 20 μm. For example, as described above at least with respect to silicon filters 100, 200, each pore of the plurality of pores 415 may have a width of about 2 μm, 4 μm, 6 μm, 8 μm, 10 μm, 12 μm, 14 μm, 16 μm, 18 μm, 20 μm, or another value therebetween. As shown in FIG. 4C, the pores 415 may comprise a circular cross-section, in which the width of each pore may otherwise be referred to as the diameter of the pore. In other examples, the cross-section of the pores 415 may be ovular, triangular, rectangular (e.g., square), or another polygonal shape.

In some embodiments, the plurality of pores 415 may be created in the filtering layer 452 such that a pitch between each pore is between 0.5:1 and 100:1 relative to the width (e.g., diameter) of the pore. For example, as described above, the pitch between pores may be about 0.5:1, 0.75:1, 1:1, 2:1, 3:1, 4:1, 5:1, 10:1, 20:1, 25:1, 50:1, 75:1, 100:1, or another ratio therebetween.

In some embodiments, the plurality of pores 415 may be created within a designated surface area of the filtering layer 452 of the silicon wafer 450. For example, the silicon wafer 450 may comprise a first surface area, and for a given silicon cell mechanoporation filter, the plurality of pores 452 may be created in a second surface area, the second surface area a portion of the first surface area. In some embodiments, the plurality of pores 415 may be created in a surface area of the filtering layer 452 comprising a width between 1 mm and 10 cm. Stated otherwise, the width of the silicon cell mechanoporation filter may be between about 1 mm and 10 cm. In some embodiments, the width of the surface area of filtering layer 452 comprising the plurality of pores 415 may be between about 1 mm-1 cm, 1-50 cm, or 1-10 cm. For example, the width of the surface area comprising the pores may be greater than or equal to about 1 mm, 5 mm, 6 mm, 10 mm, 25 mm, 50 mm, 75 mm, 1 cm, 2.5 cm, 5 cm, 7.5 cm, or 10 cm. In some embodiments, the width of the surface area comprising the pores may be greater than or equal to about 1 mm, 5 mm, 6 mm, 10 mm, 25 mm, 50 mm, 75 mm, 1 cm, 2.5 cm, 5 cm, 7.5 cm, or 10 cm. As illustrated in FIG. 4C, the silicon cell mechanoporation filter may comprise a circular shape. Thus, the width of the surface area of the filtering layer 452 which comprises the plurality of pores 415 may be the diameter.

Following etching the plurality of pores 415, the method may comprise removing (i.e., stripping) the photoresist film layer from the filtering layer 452 and cleaning the silicon wafer 450. Standard silicon wafer cleaning procedures may be used to clean the silicon wafer 450. In a non-limiting example, a 3:1 H₂SO₄:H₂O₂ piranha solution may be used to clean the silicon wafer 450.

In some embodiments, a thickness of the filtering layer 452 following creating the plurality of pores 415 may be between about 0.1 μm and 100 μm. For example, the thickness of the filtering layer 452 may be between about 0.1-50 μm, 0.1-10 μm, or 10-100 μm. In some embodiments, the thickness of the filtering layer 452 may be greater than or equal to 0.1 μm, 0.25 μm, 0.5 μm, 0.75 μm, 1 μm, 2 μm, 3 μm, 4 μm, 5 μm, 10 μm, 15 μm, 20 μm, 25 μm, 30 μm, 40 μm, 50 μm, 60 μm, 70 μm, 80 μm, or 90 μm. In some embodiments, the thickness of the filtering layer 452 may be less than or equal to 0.25 μm, 0.5 μm, 0.75 μm, 1 μm, 2 μm, 3 μm, 4 μm, 5 μm, 10 μm, 15 μm, 20 μm, 25 μm, 30 μm, 40 μm, 50 μm, 60 μm, 70 μm, 80 μm, 90 μm, or 100 μm.

As described herein, the support layer 454 may comprise a second oxide layer on a side of the support layer 454 opposite the oxide layer 456. Thus, the method may comprise, prior to etching the support structure 420 in the support layer 454, etching the second oxide layer to expose the support layer 454. In some embodiments, etching the second oxide layer may comprise reactive ion etching the second oxide layer.

As shown in FIG. 4D, the method may comprise creating a support structure 420 in the support layer 454 of the silicon wafer 450. The support structure 420 may cover at least a portion (e.g., 1%) of the filtering layer 452. In some embodiments, creating the support structure 420 may comprise applying a photoresist film layer comprising a support structure template to the support layer 454 and etching the support structure 420 in accordance with the support structure template. For example, an AZ4330 positive photoresist film layer may be applied to the support layer 454 with an exposure dose of 420 mJ. In some embodiments, the photoresist film layer may comprise a thickness between 1-10 μm. For example, the thickness of the photoresist layer may be greater than or equal to about 1 μm, 2 μm, 5 μm, or 8 μm. In some embodiments, the thickness of the photoresist layer may be less than or equal to about 2 μm, 5 μm, 8 μm, or 10 μm. In some embodiments, etching the support structure 420 may comprise deep reactive ion etching the support structure 420 through the support layer 454, as shown in FIG. 4D. FIG. 4E illustrates a top view of the support structure 420 in the silicon wafer 450.

In some embodiments, the support structure 420 may be etched to the support layer 454 such that the support structure 420 covers at least 1% of the filtering layer 452. For example, the support structure 420 may be etched such that it covers at least about 1%, 2%, 3%, 4%, 5% 6%, 7%, 8%, 9%, 10%, 15%, 20%, 25%, 30% or more of the filtering layer 452.

In some embodiments, creating the support structure 420 in the support layer 454 may comprise, for an individual silicon cell mechanoporation filter, creating a support structure 420 having a width of at least 30% of the width of the surface area of the filtering layer 452. The width of the support structure 420 may extend between a first side of the filtering layer 452 to a second side of the filtering layer 452 opposite the first side. For example, a width of the support structure may be between about 20 μm and 1 mm. In some embodiments, the width of the support structure may be between about 20-500 μm, 20-100 μm, 100 μm-1 mm, or 500 μm-1 mm. In some embodiments, the width of the support structure may be greater than or equal to about 20 μm, 25 μm, 50 μm, 75 μm, 100 μm, 125 μm, 150 μm, 175 μm, 200 μm, 250 μm, 300 μm, 350 μm, 400 μm, 450 μm, 500 μm, 600 μm, 700 μm, 800 μm, or 900 μm. In some embodiments, the width of the support structure may be less than or equal to about 25 μm, 50 μm, 75 μm, 100 μm, 125 μm, 150 μm, 175 μm, 200 μm, 250 μm, 300 μm, 350 μm, 400 μm, 450 μm, 500 μm, 600 μm, 700 μm, 800 μm, 900 μm, or 1 mm.

In some embodiments, creating the support structure 420 in the support layer 454 may comprise, for an individual silicon cell mechanoporation filter, creating at least one cross shape in the support layer 454. For example, FIG. 4E illustrates an example cross-shaped support structure 420 for a silicon cell mechanoporation filter. In the instance a plurality of silicon cell mechanoporation filters are created in the silicon wafer 450, the method may comprise creating a plurality of cross-shapes in the support layer 454, wherein at least one cross-shape is created for each individual silicon cell mechanoporation filter. Alternatively, in some embodiments, creating the support structure 420 may comprise, for an individual silicon cell mechanoporation filter, creating at least one stripe in the support layer 454 (e.g., as shown and described herein with respect to FIGS. 2K-2L).

Following etching the support structure 420, the method may comprise removing (i.e., stripping) the photoresist film layer from the support layer 454 and cleaning the silicon wafer 450. As described herein, standard silicon wafer cleaning procedures may be used to clean the silicon wafer 450.

In some embodiments, a thickness of the created support structure 420 may be between about 20 μm and 1 mm. For example, the thickness of the support structure 420 may between about 20-500 μm, 20-100 μm, 100 μm-1 mm, or 500 μm-1 mm. In some embodiments, the thickness of the support structure may be greater than or equal to about 20 μm, 25 μm, 30 μm, 40 μm, 50 μm, 60 μm, 70 μm, 75 μm, 80 μm, 90 μm, 100 μm, 125 μm, 150 μm, 175 μm, 200 μm, 250 μm, 300 μm, 350 μm, 400 μm, 450 μm, 500 μm, 600 μm, 700 μm, 750 μm, 800 μm, or 900 μm. In some embodiments, the thickness of the support structure may be less than or equal to about 25 μm, 30 μm, 40 μm, 50 μm, 60 μm, 70 μm, 75 μm, 80 μm, 90 μm, 100 μm, 125 μm, 150 μm, 175 μm, 200 μm, 250 μm, 300 μm, 350 μm, 400 μm, 450 μm, 500 μm, 600 μm, 700 μm, 750 μm, 800 μm, 900 μm, or 1 mm.

As shown in FIG. 4F, the method may comprise removing at least a portion of the oxide layer 456 to expose the plurality of pores 415 in the filtering layer 452, thereby creating one or more silicon cell mechanoporation filters 400. In some embodiments, removing the portion of the oxide layer 456 may comprise etching at least the portion of the oxide layer 456 that covers the plurality of pores 415. For example, as shown in FIG. 4F, the silicon cell mechanoporation filter 400 may comprise a remaining portion of the oxide layer 456 between the support structure 420 and the filtering layer 452. Etching the oxide layer 456 may comprise reactive ion etching the oxide layer 456.

As described herein, the above method may be understood to encompass fabricating a plurality of silicon cell mechanoporation filters. Thus, the method may comprise cutting the silicon wafer 450 into a plurality of silicon cell mechanoporation filters 400. In some embodiments, the silicon wafer 450 comprising a plurality of uncut silicon cell mechanoporation filters 400 may be cut into individual filters (i.e., chips) using standard semiconductor dicing procedures.

In some embodiments, a method for fabricating a silicon cell mechanoporation filter (e.g., silicon filter 400) may additionally or alternatively include masking a silicon wafer (e.g., silicon wafer 450) in accordance with a desired support structure layout. Then, the silicon wafer can be oxidized in the remaining areas of the silicon wafer that are not masked for the support structure. Finally, the plurality of pores can be created in the silicon wafer in the oxidized areas of the silicon wafer to create a silicon cell mechanoporation filter.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The numerical ranges disclosed inherently support any range or value within the disclosed numerical ranges, including the endpoints, even though a precise range limitation is not stated verbatim in the specification because this disclosure can be practiced throughout the disclosed numerical ranges.

### CONCLUSION

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### Exemplary Embodiments

The following embodiments are exemplary and are not intended to limit the scope of any invention described herein.

Embodiment 1. A silicon filter for cell mechanoporation, comprising:
a silicon filtering surface;
a plurality of pores extending through the silicon filtering surface and configured to perturb cell membranes as a cell mixture passes through the plurality of pores; and
a support structure disposed on a side of the silicon filtering surface and covering at least 1% of the silicon filtering surface.

Embodiment 2. The silicon filter of embodiment 1, wherein the silicon filter is configured to receive a volumetric flow rate between 0.5-500 mL/min per mm² porous surface area.

Embodiment 3. The silicon filter of embodiment 1 or 2, wherein the silicon filter is configured to receive a constant volumetric flow controlled by at least one of a peristaltic pump, a syringe pump, and a pressurized reservoir.

Embodiment 4. The silicon filter of any one of embodiments 1-3, wherein the silicon filter is configured to withstand a pressure between at least 1-50 psi.

Embodiment 5. The silicon filter of any one of embodiments 1-4, wherein the silicon filter is configured to receive a cell mixture comprising between 1.0×10⁸ and 1.0×10¹² cells.

Embodiment 6. The silicon filter of any one of embodiments 1-5, wherein the silicon filter is configured to receive a volume between 1-500 mL.

Embodiment 7. The silicon filter of any one of embodiments 1-6, wherein the silicon filter is configured to pass the cell mixture through the plurality of pores in a single run without replacing the silicon filter.

Embodiment 8. The silicon filter of any one of embodiments 1-7, wherein the silicon filter is configured to pass all of the cell mixture through the plurality of pores in no more than 30 minutes.

Embodiment 9. The silicon filter of any one of embodiments 1-8, wherein the plurality of pores comprises no more than 300,000 pores.

Embodiment 10. The silicon filter of any one of embodiments 1-9, wherein a width of each pore of the plurality of pores is between 2 μm and 20 μm.

Embodiment 11. The silicon filter of embodiment 10, wherein each pore of the plurality of pores comprises a circular cross-section, and the width of the pore is a diameter of the pore.

Embodiment 12. The silicon filter of embodiment 10 or 11, wherein a pitch between each pore of the plurality of pores is between 0.5:1 and 100:1 relative to the width of the pore.

Embodiment 13. The silicon filter of any one of embodiments 1-12, wherein each pore in the plurality of pores extends linearly through the silicon filtering surface.

Embodiment 14. The silicon filter of any one of embodiments 1-13, wherein a thickness of the silicon filtering surface is between 0.1 μm and 100 μm.

Embodiment 15. The silicon filter of any one of embodiments 1-14, wherein a thickness of the support structure is between 20 μm and 1 mm.

Embodiment 16. The silicon filter of any one of embodiments 1-15, wherein a width of the silicon filtering surface is between 1 mm and 10 cm.

Embodiment 17. The silicon filter of embodiment 16, wherein the silicon filtering surface comprises a circular shape, and the width is a diameter of the silicon filtering surface.

Embodiment 18. The silicon filter of embodiment 16 or 17, wherein a width of the support structure is at least 30% of the width of the silicon filtering surface.

Embodiment 19. The silicon filter of any one of embodiments 1-18, wherein a height of the support structure is between 20 μm and 1 mm.

Embodiment 20. The silicon filter of any one of embodiments 1-19, wherein the support structure comprises one or more supporting members disposed on the silicon filtering surface and extending in one or more directions.

Embodiment 21. The silicon filter of embodiment 20, wherein the support structure comprises a plurality of supporting members disposed on the silicon filtering surface, the plurality of supporting members together forming at least one cross shape separating a plurality of filtering windows in the silicon filtering surface.

Embodiment 22. The silicon filter of embodiment 20, wherein the support structure comprises a plurality of supporting members disposed independently as stripes on the silicon filtering surface.

Embodiment 23. The silicon filter of any one of embodiments 1-22, comprising an oxide layer disposed on the silicon filtering surface between the silicon filtering surface and the support structure.

Embodiment 24. The silicon filter of any one of embodiments 1-23, wherein the silicon filtering surface comprises silicon, silicon oxide, silicon nitride, and/or silicon carbide.

Embodiment 25. The silicon filter of any one of embodiments 1-24, wherein the support structure comprises silicon, silicon dioxide, silicon nitride, or silicon carbide.

Embodiment 26. The silicon filter of any one of embodiments 1-25, wherein the silicon filter is fabricated from a silicon wafer doped with boron, gallium, or phosphorous.

Embodiment 27. The silicon filter of any one of embodiments 1-26, wherein the silicon filter is coated with at least one of gold, silver, platinum, Teflon, polyvinylpyrrolidone, an adhesive, and a surfactant.

Embodiment 28. A system, comprising:
the silicon filter of any one of embodiments 1-27; and a cell.

Embodiment 29. The system of embodiment 28, wherein the cell is a somatic cell, an immortalized cell, a stem cell, or a derivative thereof.

Embodiment 30. The system of embodiment 28 or 29, wherein the cell is a peripheral blood mononuclear cell (PBMC), or a derivative thereof.

Embodiment 31. The system of any one of embodiments 28-30, wherein the cell is an immune cell.

Embodiment 32. The system of any one of embodiments 28-30, wherein the cell is a T cell, natural killer (NK) cell, monocyte, B cell, or dendritic cell

Embodiment 33. The system of embodiment 28 or 29, wherein the cell is a stem cell.

Embodiment 34. The system of any one of embodiments 28, 29, and 33, wherein the cell is a human stem cell.

Embodiment 35. The system of any one of embodiments 28, 29, 33, and 34, wherein the cell is an induced pluripotent stem cell, a hematopoietic cell, or a mesenchymal cell.

Embodiment 36. The system of any one of embodiments 28-35, wherein the cell is obtained, or derived, from an individual.

Embodiment 37. The system of embodiment 36, wherein the individual is a human.

Embodiment 38. A method for intracellular delivery of a payload, comprising:
passing a cell mixture comprising cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface and covering at least 1% of the silicon filtering surface; and
collecting a perturbed cell mixture comprising the cells with the payload in the cells.

Embodiment 39. The method of embodiment 38, passing the cell mixture through the plurality of pores occurs at a volumetric flow rate between 0.5-500 mL/min per mm² porous surface area.

Embodiment 40. The method of embodiment 38 or 39, wherein passing the cell mixture through the plurality of pores occurs at a constant volumetric flow rate controlled by at least one of a peristaltic pump, a syringe pump, and a pressurized reservoir.

Embodiment 41. The method of any one of embodiments 38-40, comprising, prior to passing the cell mixture through the plurality of pores, priming the plurality of pores using a cell buffer.

Embodiment 42. The method of any one of embodiments 38-41, wherein passing the cell mixture through the plurality of pores comprises passing the cell mixture through the plurality of pores in a single run without replacing the silicon cell mechanoporation filter.

Embodiment 43. The method of any one of embodiments 33-42, wherein passing the cell mixture through the plurality of pores comprises passing all of the cell mixture through the pores in no more than 30 minutes.

Embodiment 44. The method of any one of embodiments 38-43, wherein the silicon cell mechanoporation filter is configured to withstand a pressure between at least 1-50 psi.

Embodiment 45. The method of any one of embodiments 38-44, wherein the cell mixture has a volume between 1-500 mL.

Embodiment 46. The method of any one of embodiments 38-45, wherein the cell mixture comprises between 1.0×10⁸ and 1.0×10¹² cells.

Embodiment 47. The method of any one of embodiments 38-46, wherein the cell mixture comprises a somatic cell, an immortalized cell, a stem cell, or a derivative thereof.

Embodiment 48. The method of any one of embodiments 38-47, wherein the cell mixture comprises a peripheral blood mononuclear cell (PBMC), or a derivative thereof.

Embodiment 49. The method of any one of embodiments 38-48, wherein the cell mixture comprises an immune cell.

Embodiment 50. The method of any one of embodiments 38-49, wherein the cell mixture comprises a T cell, natural killer (NK) cell, monocyte, B cell, or dendritic cell.

Embodiment 51. The method of any one of embodiments 38-47, wherein the cell mixture comprises a stem cell.

Embodiment 52. The method of any one of embodiments 38-47 and 51, wherein the cell mixture comprises a human stem cell.

Embodiment 53. The method of any one of embodiments 38-47, 51, and 52, wherein the cell mixture comprises an induced pluripotent stem cell, a hematopoietic cell, or a mesenchymal cell.

Embodiment 54. The method of any one of embodiments 38-53, wherein the cell mixture comprises a cell obtained, or derived, from an individual.

Embodiment 55. The method of embodiment 54, wherein the individual is a human.

Embodiment 56. The method of any one of embodiments 38-55, comprising, prior to passing the cell mixture through the plurality of pores, mixing the payload and the cell mixture.

Embodiment 57. The method of any one of embodiments 38-56, wherein the payload comprises at least one of a polypeptide, a nucleic acid, a small molecule, a nanoparticle, and a complex thereof.

Embodiment 58. The method of any one of embodiments 38-57, wherein the payload comprises more than one of a polypeptide, a nucleic acid, a small molecule, a nanoparticle, and/or a complex thereof.

Embodiment 59. The method of any one of embodiments 38-58, wherein the plurality of pores comprises no more than 300,000 pores.

Embodiment 60. The method of any one of embodiments 38-59, wherein a width of each pore of the plurality of pores is between 2 μm and 20 μm.

Embodiment 61. The method of embodiment 60, wherein each pore of the plurality of pores comprises a circular cross-section, and the width of the pore is a diameter of the pore.

Embodiment 62. The method of embodiment 60 or 61, wherein a pitch between each pore of the plurality of pores is between 0.5:1 and 100:1 relative to the width of the pore.

Embodiment 63. The method of any one of embodiments 38-62, wherein each pore in the plurality of pores extends linearly through the silicon filtering surface.

Embodiment 64. The method of any one of embodiments 38-63, wherein a thickness of the silicon filtering surface is between 0.1 μm and 100 μm.

Embodiment 65. The method of any one of embodiments 38-64, wherein a thickness of the support structure is between 20 μm and 1 mm.

Embodiment 66. The method of any one of embodiments 38-65, wherein a width of the silicon filtering surface is between 1 mm and 10 cm.

Embodiment 67. The method of embodiment 66, wherein the silicon filtering surface comprises a circular shape, and the width is a diameter of the silicon filtering surface.

Embodiment 68. The method of embodiment 66 or 67, wherein a width of the support structure is at least 30% of the width of the silicon filtering surface.

Embodiment 69. The method of any one of embodiments 38-68, wherein a height of the support structure is between 20 μm and 1 mm.

Embodiment 70. The method of any one of embodiments 38-69, wherein the support structure comprises one or more supporting members disposed on the silicon filtering surface and extending in one or more directions.

Embodiment 71. The method of embodiment 70, wherein the support structure comprises a plurality of supporting members disposed on the silicon filtering surface, the plurality of supporting members together forming at least one cross shape separating a plurality of filtering windows in the silicon filtering surface.

Embodiment 72. The method of embodiment 70, wherein the support structure comprises a plurality of supporting members disposed independently as stripes on the silicon filtering surface.

Embodiment 73. The method of any one of embodiments 38-72, wherein the silicon cell mechanoporation filter comprises an oxide layer disposed on the silicon filtering surface between the silicon filtering surface and the support structure.

Embodiment 74. The method of any one of embodiments 38-73, wherein the silicon filtering surface comprises silicon, silicon oxide, silicon nitride, and/or silicon carbide.

Embodiment 75. The method of any one of embodiments 38-74, wherein the support structure comprises silicon, silicon dioxide, silicon nitride, or silicon carbide.

Embodiment 76. The method of any one of embodiments 38-75, wherein the silicon cell mechanoporation filter is fabricated from a silicon wafer doped with boron, gallium, or phosphorous.

Embodiment 77. The method of any one of embodiments 38-76, wherein the silicon cell mechanoporation filter is coated with at least one of gold, silver, platinum, Teflon, polyvinylpyrrolidone, an adhesive, and a surfactant.

Embodiment 78. A cell mechanoporation system, comprising:
a filter holder fluidly connectable to a cell mixture source;
one or more silicon cell mechanoporation filters disposed in the filter holder, the one or more silicon cell mechanoporation filters comprising:
   a silicon filtering surface;
   a plurality of pores extending through the silicon filtering surface and configured to perturb cell membranes as a cell mixture from the cell mixture source passes through the plurality of pores; and
   a support structure disposed on a side of the silicon filtering surface and covering at least 1% of the silicon filtering surface;
an output reservoir fluidly connected to the filter holder to collect a perturbed cell mixture; and
a pump configured to move the cell mixture through the one or more silicon cell mechanoporation filters and into the output reservoir.

Embodiment 79. The system of embodiment 78, wherein the one or more silicon cell mechanoporation filters are configured to receive a volumetric flow rate between 0.5-500 mL/min per mm² porous surface area.

Embodiment 80. The system of embodiment 78 or 79, wherein the cell mechanoporation system is operable at a constant volumetric flow rate controlled by at least one of the pump and a pressurized reservoir.

Embodiment 81. The system of any one of embodiments 78-80, wherein the one or more silicon cell mechanoporation filters are configured to withstand a pressure between at least 1-50 psi.

Embodiment 82. The system of any one of embodiments 78-81, wherein the one or more silicon cell mechanoporation filters are configured to receive a volume between 1-500 mL.

Embodiment 83. The system of any one of embodiments 78-82, wherein the one or more silicon cell mechanoporation filters comprises a single silicon cell mechanoporation filter.

Embodiment 84. The system of any one of embodiments 78-83, wherein the one or more silicon cell mechanoporation filters are configured to pass the cell mixture through the plurality of pores in a single run without replacing the one or more silicon cell mechanoporation filters.

Embodiment 85. The system of any one of embodiments 78-84, wherein the one or more silicon cell mechanoporation filters are configured to pass the cell mixture through the plurality of pores in no more than 30 minutes.

Embodiment 86. The system of any one of embodiments 78-85, wherein the pump comprises a peristaltic pump or a syringe pump.

Embodiment 87. The system of any one of embodiments 78-86, wherein the pump is of a fluid delivery system.

Embodiment 88. The system of embodiment 87, wherein the fluid delivery system comprises a filtration system, an elutriation system, a cell washing system, an isolation system, or a cell therapy production system.

Embodiment 89. The system of any one of embodiments 78-88, comprising the cell mixture source.

Embodiment 90. The system of any one of embodiments 78-89, comprising a payload source fluidly connectable to the filter holder and containing a payload.

Embodiment 91. The system of embodiment 90, wherein the payload source is fluidly connected to the cell mixture source to receive the cell mixture from the cell mixture source.

Embodiment 92. The system of any one of embodiments 78-89, wherein the cell mixture source comprises a payload.

Embodiment 93. The system of any one of embodiments 90-92, wherein the payload comprises at least one of a polypeptide, a nucleic acid, a small molecule, a nanoparticle, and a complex thereof.

Embodiment 94. The system of any one of embodiments 90-93, wherein the payload comprises more than one of a polypeptide, a nucleic acid, a small molecule, a nanoparticle, and/or a complex thereof.

Embodiment 95. The system of any one of embodiments 78-94, wherein the one or more silicon cell mechanoporation filters are configured to receive a cell mixture comprising between 1.0×10⁸ and 1.0×10¹² cells.

Embodiment 96. The system of any one of embodiments 78-95, wherein the cell mixture comprises a somatic cell, an immortalized cell, a stem cell, or a derivative thereof.

Embodiment 97. The system of any one of embodiments 78-96, wherein the cell mixture comprises a peripheral blood mononuclear cell (PBMC), or a derivative thereof.

Embodiment 98. The system of any one of embodiments 78-97, wherein the cell mixture comprises an immune cell.

Embodiment 99. The system of any one of embodiments 78-98, wherein the cell mixture comprises a T cell, natural killer (NK) cell, monocyte, B cell, or dendritic cell.

Embodiment 100. The system of any one of embodiments 78-96, wherein the cell mixture comprises a stem cell.

Embodiment 101. The system of any one of embodiments 78-96 and 100, wherein the cell mixture comprises a human stem cell.

Embodiment 102. The system of any one of embodiments 78-96, 100, and 101, wherein the cell mixture comprises an induced pluripotent stem cell, a hematopoietic cell, or a mesenchymal cell.

Embodiment 103. The system of any one of embodiments 78-102, wherein the cell mixture comprises a cell obtained, or derived, from an individual.

Embodiment 104. The system embodiment 103, wherein the individual is a human.

Embodiment 105. The system of any one of embodiments 78-104, wherein the plurality of pores comprises no more than 300,000 pores.

Embodiment 106. The system of any one of embodiments 78-105, wherein a width of each pore of the plurality of pores is between 2 μm and 20 μm.

Embodiment 107. The system of embodiment 106, wherein each pore of the plurality of pores comprises a circular cross-section, and the width of the pore is a diameter of the pore.

Embodiment 108. The system of embodiment 106 or 107, wherein a pitch between each pore of the plurality of pores is between 0.5:1 and 100:1 relative to the width of the pore.

Embodiment 109. The system of any one of embodiments 78-108, wherein each pore in the plurality of pores extends linearly through the silicon filtering surface.

Embodiment 110. The system of any one of embodiments 78-109, wherein a thickness of the silicon filtering surface is between 0.1 μm and 100 μm.

Embodiment 111. The system of any one of embodiments 78-110, wherein a thickness of the support structure is between 20 μm and 1 mm.

Embodiment 112. The system of any one of embodiments 78-111, wherein a width of the silicon filtering surface is between 1 mm and 10 cm.

Embodiment 113. The system of embodiment 112, wherein the silicon filtering surface comprises a circular shape, and the width is a diameter of the silicon filtering surface.

Embodiment 114. The system of embodiment 112 or 113, wherein a width of the support structure is at least 30% of the width of the silicon filtering surface.

Embodiment 115. The system of any one of embodiments 78-114, wherein a height of the support structure is between 20 μm and 1 mm.

Embodiment 116. The system of any one of embodiments 78-115, wherein the support structure comprises one or more supporting members disposed on the silicon filtering surface and extending in one or more directions.

Embodiment 117. The system of embodiment 116, wherein the support structure comprises a plurality of supporting members disposed on the silicon filtering surface, the plurality of supporting members together forming at least one cross shape separating a plurality of filtering windows in the silicon filtering surface.

Embodiment 118. The system of embodiment 116, wherein the support structure comprises a plurality of supporting members disposed independently as stripes on the silicon filtering surface.

Embodiment 119. The system of any one of embodiments 78-118, wherein the one or more silicon cell mechanoporation filters comprise an oxide layer disposed on the silicon filtering surface between the silicon filtering surface and the support structure.

Embodiment 120. The system of any one of embodiments 78-119, wherein the silicon filtering surface comprises silicon, silicon oxide, silicon nitride, and/or silicon carbide.

Embodiment 121. The system of any one of embodiments 78-120, wherein the support structure comprises silicon, silicon dioxide, silicon nitride, or silicon carbide.

Embodiment 122. The system of any one of embodiments 78-121, wherein the one or more silicon cell mechanoporation filters are fabricated from a silicon wafer doped with boron, gallium, or phosphorous.

Embodiment 123. The system of any one of embodiments 78-122, wherein the one or more silicon cell mechanoporation filters are coated with at least one of gold, silver, platinum, Teflon, polyvinylpyrrolidone, an adhesive, and a surfactant.

Embodiment 124. A method of fabricating a silicon cell mechanoporation filter, comprising:
creating a plurality of pores in a filtering layer of a silicon wafer, the silicon wafer comprising a support layer, an oxide layer on a side of the support layer, and the filtering layer on a side of the oxide layer opposite the support layer;
creating a support structure in the support layer of the silicon wafer, wherein the support structure covers at least 1% of the filtering layer; and
removing at least a portion of the oxide layer to expose the plurality of pores in the filtering layer and create one or more silicon cell mechanoporation filters.

Embodiment 125. The method of embodiment 124, comprising cutting the silicon wafer into a plurality of silicon cell mechanoporation filters.

Embodiment 126. The method of embodiment 124 or 125, wherein creating the plurality of pores in the filtering layer comprises applying a photoresist film layer comprising a pore template to the filtering layer and etching the plurality of pores in accordance with the pore template.

Embodiment 127. The method of embodiment 126, wherein etching the plurality of pores through the filtering layer comprises deep reactive ion etching the plurality of pores linearly through the filtering layer and to the oxide layer.

Embodiment 128. The method of embodiment 126 or 127, comprising, following etching, removing the photoresist film layer from the filtering layer, and cleaning the silicon wafer.

Embodiment 129. The method of any one of embodiments 124-128, wherein creating the plurality of pores comprises, for an individual silicon cell mechanoporation filter, creating no more than 300,000 pores in the filtering layer.

Embodiment 130. The method of any one of embodiments 124-129, wherein creating the plurality of pores in the filtering layer comprise creating a plurality of pores having a width between 2 μm and 20 μm.

Embodiment 131. The method of embodiment 130, wherein creating the plurality of pores in the filtering layer comprise creating a plurality of pores having a circular cross-section, the width of each pore being a diameter of the pore.

Embodiment 132. The method of embodiment 130 or 131, wherein creating the plurality of pores in the filtering layer comprises creating a plurality of pores with a pitch between each pore between 0.5:1 and 100:1 relative to the width of the pore.

Embodiment 133. The method of any one of embodiments 124-132, wherein creating the plurality of pores in the filtering layer comprises, for an individual silicon cell mechanoporation filter, creating the plurality of pores within a surface area of the filtering layer comprising a width between 1 mm and 10 cm.

Embodiment 134. The method of embodiment 133, wherein the surface area of the filtering layer comprises a circular shape, and the width is a diameter of the surface area of the filtering layer.

Embodiment 135. The method of any one of embodiments 124-134, wherein creating the support structure in the support layer comprises applying a photoresist film layer comprising a support structure template to the support layer and etching the support structure in accordance with the support structure template.

Embodiment 136. The method of embodiment 135, wherein etching the support structure comprises deep reactive ion etching the support structure through the support layer.

Embodiment 137. The method of embodiment 135 or 136, wherein the support layer comprises a second oxide layer on a side of the support layer opposite the oxide layer, and the method comprises, prior to etching the support structure, etching the second oxide layer to expose the support layer.

Embodiment 138. The method of embodiment 137, wherein etching the second oxide layer comprise reactive ion etching the second oxide layer.

Embodiment 139. The method of any one of embodiments 135-138, comprising, following etching the support structure, removing the photoresist film layer from the support layer, and cleaning the silicon wafer.

Embodiment 140. The method of any one of embodiments 124-139, wherein creating the support structure in the support layer comprises, for an individual silicon cell mechanoporation filter, creating a support structure having a width of at least 30% of a width of a surface area of the filtering layer.

Embodiment 141. The method of any one of embodiments 124-140, wherein creating the support structure in the support layer comprises creating a support structure having a width between 20 μm and 1 mm.

Embodiment 142. The method of any one of embodiments 124-141, wherein creating the support structure in the support layer comprises, for an individual silicon cell mechanoporation filter, creating at least one cross shape in the support layer.

Embodiment 143. The method of any one of embodiments 124-141, wherein creating the support structure in the support layer comprises, for an individual silicon cell mechanoporation filter, creating at least one stripe in the support layer.

Embodiment 144. The method of any one of embodiments 124-143, wherein a thickness of the filtering layer after creating the plurality of pores is between 0.1 μm and 100 μm.

Embodiment 145. The method of any one of embodiments 124-144, wherein a thickness of the created support structure is between 20 μm and 1 mm.

Embodiment 146. The method of any one of embodiments 124-145, wherein removing at least a portion of the oxide layer comprises etching at least the portion of the oxide layer covering the plurality of pores.

Embodiment 147. The method of embodiment 146, wherein etching the at least a portion of the oxide layer comprises reactive ion etching the at least a portion of the oxide layer.

Embodiment 148. The method of any one of embodiments 124-147, wherein the silicon wafer comprises silicon, silicon oxide, silicon nitride, and/or silicon carbide.

Embodiment 149. The method of any one of embodiments 124-148, wherein the silicon wafer is doped with boron, gallium, or phosphorous.

Embodiment 150. The method of any one of embodiments 124-149, comprising, prior to creating the plurality of pores, doping the silicon wafer with boron, gallium, or phosphorous.

Embodiment 151. The method of any one of embodiments 124-150, wherein the silicon wafer is coated with at least one of gold, silver or platinum.

Embodiment 152. The method of any one of embodiments 124-151, comprising coating the one or more silicon cell mechanoporation filters with at least one of gold, silver, platinum, Teflon, polyvinylpyrrolidone, an adhesive, and a surfactant.

Embodiment 153. The method of any one of embodiments 124-136, wherein the one or more silicon cell mechanoporation filters are configured to receive a volumetric flow rate between 0.5-500 mL/min per mm² porous surface area.

Embodiment 154. The method of any one of embodiments 124-153, wherein the one or more silicon cell mechanoporation filters are configured to receive a constant volumetric flow controlled by at least one of a peristaltic pump, a syringe pump, and a pressurized reservoir.

Embodiment 155. The method of any one of embodiments 124-154, wherein the one or more silicon cell mechanoporation filters are configured to withstand a pressure between at least 1-50 psi.

Embodiment 156. The method of any one of embodiments 124-155, wherein the one or more silicon cell mechanoporation filters are configured to receive a volume between 1-500 mL.

Embodiment 157. The method of any one of embodiments 124-156, wherein the one or more silicon cell mechanoporation filters are configured to receive a cell mixture comprising between 1.0×10⁸ and 1.0×10¹² cells.

Embodiment 158. The method of any one of embodiments 124-157, wherein the one or more silicon cell mechanoporation filters are configured to pass a cell mixture through the plurality of pores in a single run without replacing the one or more silicon cell mechanoporation filters.

Embodiment 159. The method of any one of embodiments 124-158, wherein the one or more silicon cell mechanoporation filters are configured to pass all of a cell mixture through the plurality of pores in no more than 30 minutes.

Embodiment 160. A method for intracellular delivery of a payload, comprising:
passing a cell mixture comprising at least 1.0×10⁸ cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface that covers at least a portion of the silicon filtering surface; and
collecting a perturbed cell mixture comprising the cells with the payload in the cells.

Embodiment 161. The method of embodiment 160, wherein the cell mixture comprises between 1.0×10⁸ and 1.0×10¹² cells.

Embodiment 162. The method of embodiment 160 or 161, wherein the support structure covers at least 1% of the silicon filtering surface.

Embodiment 163. The method of any one of embodiments 160-162, wherein passing the cell mixture through the plurality of pores occurs at a volumetric flow rate between 0.5-500 mL/min per mm² porous surface area.

Embodiment 164. The method of any one of embodiments 160-163, wherein passing the cell mixture through the plurality of pores occurs at a constant volumetric flow rate controlled by at least one of a peristaltic pump, a syringe pump, and a pressurized reservoir.

Embodiment 165. The method of any one of embodiments 160-164, comprising, prior to passing the cell mixture through the plurality of pores, priming the plurality of pores using a cell buffer.

Embodiment 166. The method of any one of embodiments 160-165, wherein passing the cell mixture through the plurality of pores comprises passing the cell mixture through the plurality of pores in a single run without replacing the silicon cell mechanoporation filter.

Embodiment 167. The method of any one of embodiments 160-166, wherein passing the cell mixture through the plurality of pores comprises passing all of the cell mixture through the pores in no more than 30 minutes.

Embodiment 168. The method of any one of embodiments 160-167, wherein the silicon cell mechanoporation filter is configured to withstand a pressure between at least 1-50 μsi.

Embodiment 169. The method of any one of embodiments 160-168, wherein the cell mixture has a volume between 1-500 mL.

Embodiment 170. The method of any one of embodiments 160-169, wherein the cell mixture comprises a somatic cell, an immortalized cell, a stem cell, or a derivative thereof.

Embodiment 171. The method of any one of embodiments 160-170, wherein the cell mixture comprises a peripheral blood mononuclear cell (PBMC), or a derivative thereof.

Embodiment 172. The method of any one of embodiments 160-171, wherein the cell mixture comprises an immune cell.

Embodiment 173. The method of any one of embodiments 160-172, wherein the cell mixture comprises a T cell, natural killer (NK) cell, monocyte, B cell, or dendritic cell.

Embodiment 174. The method of any one of embodiments 160-170, wherein the cell mixture comprises a stem cell.

Embodiment 175. The method of any one of embodiments 160-170 and 174, wherein the cell mixture comprises a human stem cell.

Embodiment 176. The method of any one of embodiments 160-170, 174, and 175, wherein the cell mixture comprises an induced pluripotent stem cell, a hematopoietic cell, or a mesenchymal cell.

Embodiment 177. The method of any one of embodiments 160-176, wherein the cell mixture comprises a cell obtained, or derived, from an individual.

Embodiment 178. The method of embodiment 177, wherein the individual is a human.

Embodiment 179. The method of any one of embodiments 160-178, comprising, prior to passing the cell mixture through the plurality of pores, mixing the payload and the cell mixture.

Embodiment 180. The method of any one of embodiments 160-179, wherein the payload comprises at least one of a polypeptide, a nucleic acid, a small molecule, a nanoparticle, and a complex thereof.

Embodiment 181. The method of any one of embodiments 160-180, wherein the payload comprises more than one of a polypeptide, a nucleic acid, a small molecule, a nanoparticle, and/or a complex thereof.

Embodiment 182. The method of any one of embodiments 160-181, wherein the plurality of pores comprises no more than 300,000 pores.

Embodiment 183. The method of any one of embodiments 160-182, wherein a width of each pore of the plurality of pores is between 2 μm and 20 μm.

Embodiment 184. The method of embodiment 183, wherein each pore of the plurality of pores comprises a circular cross-section, and the width of the pore is a diameter of the pore.

Embodiment 185. The method of embodiment 183 or 184, wherein a pitch between each pore of the plurality of pores is between 0.5:1 and 100:1 relative to the width of the pore.

Embodiment 186. The method of any one of embodiments 160-185, wherein each pore in the plurality of pores extends linearly through the silicon filtering surface.

Embodiment 187. The method of any one of embodiments 160-186, wherein a thickness of the silicon filtering surface is between 0.1 μm and 100 μm.

Embodiment 188. The method of any one of embodiments 160-187, wherein a thickness of the support structure is between 20 μm and 1 mm.

Embodiment 189. The method of any one of embodiments 160-188, wherein a width of the silicon filtering surface is between 1 mm and 10 cm.

Embodiment 190. The method of embodiment 189, wherein the silicon filtering surface comprises a circular shape, and the width is a diameter of the silicon filtering surface.

Embodiment 191. The method of embodiment 189 or 190, wherein a width of the support structure is at least 30% of the width of the silicon filtering surface.

Embodiment 192. The method of any one of embodiments 160-191, wherein a height of the support structure is between 20 μm and 1 mm.

Embodiment 193. The method of any one of embodiments 160-192, wherein the support structure comprises one or more supporting members disposed on the silicon filtering surface and extending in one or more directions.

Embodiment 194. The method of embodiment 193, wherein the support structure comprises a plurality of supporting members disposed on the silicon filtering surface, the plurality of supporting members together forming at least one cross shape separating a plurality of filtering windows in the silicon filtering surface.

Embodiment 195. The method of embodiment 193, wherein the support structure comprises a plurality of supporting members disposed independently as stripes on the silicon filtering surface.

Embodiment 196. The method of any one of embodiments 160-195, wherein the silicon cell mechanoporation filter comprises an oxide layer disposed on the silicon filtering surface between the silicon filtering surface and the support structure.

Embodiment 197. The method of any one of embodiments 160-196, wherein the silicon filtering surface comprises silicon, silicon oxide, silicon nitride, and/or silicon carbide.

Embodiment 198. The method of any one of embodiments 160-197, wherein the support structure comprises silicon, silicon dioxide, silicon nitride, or silicon carbide.

Embodiment 199. The method of any one of embodiments 160-198, wherein the silicon cell mechanoporation filter is fabricated from a silicon wafer doped with boron, gallium, or phosphorous.

Embodiment 200. The method of any one of embodiments 160-199, wherein the silicon cell mechanoporation filter is coated with at least one of gold, silver, platinum, Teflon, polyvinylpyrrolidone, an adhesive, and a surfactant.

Embodiment 201. The silicon filter of any one of embodiments 5-27, wherein the volume is such that a residence time of the cell mixture after passing through the plurality of pores is less than 30 seconds.

Embodiment 202. The method of any one of embodiments 45-77, wherein the volume is such that a residence time of the cell mixture after passing through the plurality of pores is less than 30 seconds.

Embodiment 203. The system of any one of embodiments 82-123, wherein the volume is such that a residence time of the cell mixture after passing through the plurality of pores is less than 30 seconds.

Embodiment 204. The method of any one of embodiments 156-159, wherein the volume is such that a residence time of the cell mixture after passing through the plurality of pores is less than 30 seconds.

Embodiment 205. The method of any one of embodiments 169-200, wherein the volume is such that a residence time of the cell mixture after passing through the plurality of pores is less than 30 seconds.

Embodiment 206. The silicon filter of any one of embodiments 2-27, wherein a pressure gradient across the silicon filtering surface at the volumetric flow rate is less than 20 μsi.

Embodiment 207. The method of any one of embodiments 39-77, wherein a pressure gradient across the silicon filtering surface at the volumetric flow rate is less than 20 psi.

Embodiment 208. The system of any one of embodiments 79-123, wherein a pressure gradient across the silicon filtering surface at the volumetric flow rate is less than 20 psi.

Embodiment 209. The method of any one of embodiments 153-159, wherein a pressure gradient across the silicon filtering surface at the volumetric flow rate is less than 20 μsi.

Embodiment 210. The method of any one of embodiments 163-200, wherein a pressure gradient across the silicon filtering surface at the volumetric flow rate is less than 20 μsi.

### Examples

Experimentation demonstrating the ability of various silicon filters described herein to withstand pressure and flow rate conditions induced by upstream fluid delivery systems is provided herein with respect to several examples.

Methods: Various filter schematics were tested in a closed system according to the following set-up. A fluid source comprising tap water was fluidly connected to a filter holder containing the silicon filter. Tap water was used because its composition is understood to be representative of a sample comprising particles that cause fouling over time. The fluid source and the filter holder were fluidly connected via tubing. Between the fluid source and the filter holder containing the filter, a pump (i.e., a retentate pump) and a pressure sensor were disposed (in that order, although an alternative order between the fluid source and the filter holder may be envisaged and is understood to be encompassed in the scope of the disclosure provided herein). One or more aforementioned components, including the fluid source, at least a portion of the tubing, and the pump may be of a fluid delivery system (e.g., a cell processing system). Alternatively, the methods described herein may be performed using a custom tabletop apparatus configured to receive the sample from a fluid source and drive the sample through the filter at a controlled rate. Following the silicon filter, an output reservoir (e.g., a collection bag) was fluidly connected to the filter holder.

Eight silicon filter schematics from those illustrated in FIGS. 2A-2L (specifically, FIGS. 2C-2J) were selected for testing, indicated below in Table 1. The tested filters were embodied in a chip sized 10×10 mm, and the filtering surface had a circular shape with a 7 mm diameter. The surface area of the filtering surface of the 10×10 mm filters was 38.48 mm². The surface area of the filtering surface is the area of the silicon filtering surface remaining after creating the support structure. Filter 200c illustrated in FIG. 2C was also tested in a 5×5 mm chip with a 3.5 mm diameter circular-shaped filtering surface. The surface area of the filtering surface of the 5×5 mm filter was 9.62 mm². All pores of the filters, regardless of the size of the filter, were 5 μm in diameter.

The silicon filters were tested against an existing cell mechanoporation filter, 5×5 mm Aquamarijn filter (also characterized in Table 1). The filtering surface of the Aquamarijn filter has a rectangular (i.e., square) shape, and thus has a surface area of about 12.5 mm². The Aquamarijn filter includes a striped support structure covering about 50% of the filtering surface (e.g., closely resembling the schematic illustrated in FIG. 2L). Like the filters 200c-200j above, the diameter of the pores of the Aquamarijn filter tested was 5 μm.

A flow rate of 50 mL/min was induced on each filter to determine whether the filter could withstand pressure induced by the upstream pump, or fluid delivery system, at the constant 50 mL/min flow rate. Each filter was tested until a pressure of about 35-40 psi accumulated in the system, or until the filter broke.

TABLE 1
Filter schematics tested

| | | Support | | | |
|---|---|---|---|---|---|
| | | Structure | % | Estimated | |
| Pressure | | Area | Support | # of | Porosity |
| Graph | Filter | (mm²) | Structure | Pores | (%) |
| FIG. 5A | Aquamarijn 5- | 12.5 | 50% | N/A | N/A |
| | μm striped | | | | |
| FIG. 5B | 200c (FIG. 2C)- | 1.36 | 14.14% | 20,535 | 4.19% |
| | 5 × 5 mm | | | | |
| FIG. 5C | 200c (FIG. 2C)- | 2.7 | 7.07% | 99,581 | 4.22% |
| | 10 × 10 mm | | | | |
| FIG. 5D | 200d (FIG. 2D) | 5.2 | 13.41% | 90,017 | 4.24% |
| FIG. 5E | 200e (FIG. 2E) | 4.0 | 10.34% | 94,628 | 4.39% |
| FIG. 5F | 200f (FIG. 2F) | 5.2 | 13.51% | 89,069 | 4.23% |
| FIG. 5G | 200g (FIG. 2G) | 6.3 | 16.47% | 86,589 | 4.09% |
| FIG. 5H | 200h (FIG. 2H) | 2.9 | 7.44% | 98,691 | 4.53% |
| FIG. 5I | 200i (FIG. 2I) | 3.3 | 8.66% | 97,043 | 4.47% |
| FIG. 5J | 200j (FIG. 2J) | 4.3 | 11.08% | 94,647 | 4.35% |

Table 1 maps each of the graphs illustrated in FIGS. 5A-5J to the corresponding tested filter schematic. For each filter design, the surface area of the support structure, the ratio of the surface area of the support structure to the surface area of the filtering surface (e.g., “% support structure”), the estimated number of pores, and the porosity is provided. The estimated number of pores and porosity were not available for the Aquamarijn filter, and the % support structure and support structure surface area were estimated using a visual analysis. These parameters can be analyzed to understand the effect of filter design in withstanding the pressure conditions induced by the constant flow rate.

With reference to the filter designs of interest (filters 200c-200j illustrated in FIGS. 2C-2J), as shown in Table 1, the estimated number of pores may be between about 50,000-200,000 pores, 50,000-100,000 pores, or about 75,000-100,000 pores. In some embodiments, the number of pores may be greater than or equal to about 50,000, 60,000, 75,000, 80,000, 90,000, 95,000, or 99,000 pores. In some embodiments, the number of pores may be less than or equal to 200,000, 175,000, 150,000, 125,000, 110,000, 100,000, 99,000, 95,000, or 90,000 pores.

As shown in Table 1, the porosity of the filter (e.g., filters 200c-200j) may be between about 1-20%, 1-15%, 1-10%, or 1-5%. The porosity is defined as the amount (expressed as a percentage) of the filtering surface that is porous. The porosity of the filter may dictate the amount of support needed from the support structure to withstand flow through the filter and the pressure conditions induced by connected fluid delivery systems. For example, a higher porosity may require a larger support structure (i.e., a greater ratio between support structure and porous surface area). A lower porosity may require less support structure (i.e., a smaller ratio between support structure and porous surface area). In some embodiments, the porosity of the filter may be greater than or equal to about 1%, 1.5%, 2%, 2.5%, 3%, 3.5%, 4%, 4.25%, 4.5%, 4.75%, or 5%. In some embodiments, the porosity of the filter may be less than or equal to 20%, 18%, 15%, 12%, 10%, 8%, 5%, 4.75%, 4.5%, 4.25%, or 4%.

In some embodiments, the ratio of the surface area of the support structure to the surface area of the filtering surface for each of the filters of interest (filters 200c-200j) may be between about 1-20%, 1-15%, 1-10%, or 5-10%. In some embodiments, ratio of the surface area of the support structure to the surface area of the filtering surface may be greater than or equal to about 1%, 2%, 5%, 7%, 8%, 10%, 12%, 15%, or 18%. In some embodiments, the ratio of the surface area of the support structure to the surface area of the filtering surface may be less than or equal to about 5%, 8%, 10%, 12%, 15%, 18%, or 20%.

Results: All but the 5×5 mm filter 200c, illustrated in FIG. 2C, withstood the pressure conditions within the closed system at the volumetric flow rate of 50 mL/min. In each of the graphs illustrated in FIGS. 5A-5J, several data points over a short duration of time are observed because the upstream fluid delivery system used to generate the results operates with a pulsatile flow, thus generating a cyclic pattern in the captured data points. In these tests, the pulsatile flow was minimized (as is clear from the minimal variation in the cyclic pattern) using pressure dampeners or other mechanisms. More specific results for each of the seven tested silicon filters are summarized below.

Aquamarijn 5×5 mm filter: As shown in FIG. 5A, within less than about 1 minute (e.g., about 50 seconds), the Aquamarijn filter can reach a pressure of about 40 psi. However, as the pressure in the system increases over the illustrated duration of time, the fluctuations in measured pressure appear to increase. The performance of the Aquamarijn filter demonstrated in FIG. 5A can suggest that the filter may not be suitable to filter large sample volumes with millions of cells (e.g., provided by upstream cell processing systems) without rapidly clogging. Based on the performance of the filter, it may be preferable to achieve a minimally supported filtering surface that also can withstand breakage at the desired pressure conditions.

5×5 mm Filter 200c (FIG. 2C): As shown in FIG. 5B, within about 1.5 minutes (e.g., about 90 seconds), the 5×5 mm filter 200c filter can reach a pressure of about 40 psi. However, as the pressure in the system increases over the illustrated duration of time, the fluctuations in measured pressure appear to increase. The performance of the 5×5 mm filter demonstrated in FIG. 5B may suggest that the filter may not be suitable to filter large sample volumes with millions of cells (e.g., provided by upstream cell processing systems) without rapidly clogging. This may be due at least in part to the number of pores in the filtering surface compared to the total available surface area of the filtering surface.

10×10 mm Filter 200c (FIG. 2C): As mentioned above and illustrated in FIG. 5C, at a flow rate of 50 mL/min, the filter 200c can withstand a pressure of at least about 20 psi before breaking. In some embodiments, the silicon filter 200c can withstand a pressure of between about 1-20 psi, such as at least about 12 psi, 15 psi, 18 psi, or 20 psi. Although filter 200c may not have withstood the same or a similar amount of pressure (e.g., between about 35-40 psi) as the other filter designs, it is contemplated that the design of filter 200c may nonetheless perform well (i.e., may receive flow and filter the sample at a desired flow rate without breaking) in a research-scale system. This conclusion may be related to the ratio of the surface area of the support structure to the surface area of the filtering surface. It is contemplated that the pressure conditions induced by a research-scale system may be lower than that of a clinical scale system, and thus a lower ratio of the surface area of the support structure to the surface area of the filtering surface may be sufficient.

Filter 200d (FIG. 2D): As shown in FIG. 5D, at a flow rate of 50 mL/min, the filter 200d can withstand a pressure of at least about 35 psi without breaking. In some embodiments, the silicon filter 200d can withstand a pressure of between about 2-40 psi, 2-35 psi, 2-30 psi, 5-40 psi, 5-35 psi, 5-30 psi, 8-40 psi, 8-35 psi, or 8-30 psi. In some embodiments, the silicon filter 200d can withstand a pressure of at least about 30 psi, 35 psi, or 40 psi. The filter 200d can reach the pressure of about 35 psi within less than 10 minutes (e.g., about 8.5 min). In some embodiments, the filter 200d can reach a pressure of about 35 psi within about 10 min, 9.5 min, 9 min, 8.5 min, 8 min, 7.5 min, or 7 min.

Filter 200e (FIG. 2E): As shown in FIG. 5E, at a flow rate of 50 mL/min, the filter 200e can withstand a pressure of at least about 35 psi without breaking. In some embodiments, the silicon filter 200e can withstand a pressure of between about 2-40 psi, 2-35 psi, 2-30 psi, 5-40 psi, 5-35 psi, 5-30 psi, 8-40 psi, 8-35 psi, or 8-30 psi. In some embodiments, the silicon filter 200e can withstand a pressure of at least about 30 psi, 35 psi, or 40 psi. The filter 200e can reach the pressure of about 35 psi within less than 8 minutes (e.g., about 6.5 min). In some embodiments, the filter 200e can reach a pressure of about 35 psi within about 8 min, 7.5 min, 7 min, 6.5 min, 6 min, 5.5 min, or 5 min.

Filter 200f (FIG. 2F): As shown in FIG. 5F, at a flow rate of 50 mL/min, the filter 200f can withstand a pressure of at least about 40 psi without breaking. In some embodiments, the silicon filter 200f can withstand a pressure of between about 2-40 psi, 2-35 psi, 2-30 psi, 5-40 μsi, 5-35 psi, 5-30 psi, 8-40 psi, 8-35 psi, or 8-30 psi. In some embodiments, the silicon filter 200f can withstand a pressure of at least about 30 psi, 35 psi, or 40 psi. The filter 200f can reach the pressure of about 35 psi within less than 10 minutes (e.g., about 8.5 min). In some embodiments, the filter 200f can reach a pressure of about 40 psi within about 10 min, 9.5 min, 9 min, 8.5 min, 8 min, 7.5 min, or 7 min.

Filter 200g (FIG. 2G): As shown in FIG. 5G, at a flow rate of 50 mL/min, the filter 200g can withstand a pressure of at least about 35 psi without breaking. In some embodiments, the silicon filter 200g can withstand a pressure of between about 2-40 psi, 2-35 psi, 2-30 psi, 5-40 psi, 5-35 psi, 5-30 psi, 8-40 psi, 8-35 psi, or 8-30 psi. In some embodiments, the silicon filter 200g can withstand a pressure of at least about 30 psi, 35 psi, or 40 psi. The filter 200g can reach the pressure of about 35 psi within less than 6 minutes (e.g., about 4.5 min). In some embodiments, the filter 200g can reach a pressure of about 35 psi within about 6 min, 5.5 min, 5 min, 4.5 min, 4 min, 3.5 min, or 3 min.

Filter 200h (FIG. 2H): As shown in FIG. 5H, at a flow rate of 50 mL/min, the filter 200h can withstand a pressure of at least about 40 psi without breaking. In some embodiments, the silicon filter 200h can withstand a pressure of between about 2-40 psi, 2-35 psi, 2-30 psi, 5-40 psi, 5-35 psi, 5-30 psi, 8-40 psi, 8-35 psi, or 8-30 psi. In some embodiments, the silicon filter 200h can withstand a pressure of at least about 30 psi, 35 psi, or 40 psi. The filter 200h can reach the pressure of about 40 psi within less than 7 minutes (e.g., about 6 min). In some embodiments, the filter 200h can reach a pressure of about 40 psi within about 7 min, 6.5 min, 6 min, 5.5 min, or 5 min.

Filter 200i (FIG. 2I): As shown in FIG. 5I, at a flow rate of 50 mL/min, the filter 200i can withstand a pressure of at least about 40 psi without breaking. In some embodiments, the silicon filter 200g can withstand a pressure of between about 2-40 psi, 2-35 psi, 2-30 psi, 5-40 μsi, 5-35 psi, 5-30 psi, 8-40 psi, 8-35 psi, or 8-30 psi. In some embodiments, the silicon filter 200g can withstand a pressure of at least about 30 psi, 35 psi, or 40 psi. The filter 200i can reach the pressure of about 40 psi within less than 7 minutes (e.g., about 6 min). In some embodiments, the filter 200i can reach a pressure of about 40 psi within about 7 min, 6.5 min, 6 min, 5.5 min, or 5 min.

Filter 200j (FIG. 2J): As shown in FIG. 5J, at a flow rate of 50 mL/min, the filter 200j can withstand a pressure of at least about 35 psi without breaking. In some embodiments, the silicon filter 200j can withstand a pressure of between about 2-40 psi, 2-35 psi, 2-30 psi, 5-40 μsi, 5-35 psi, 5-30 psi, 8-40 psi, 8-35 psi, or 8-30 psi. In some embodiments, the silicon filter 200j can withstand a pressure of at least about 30 psi, 35 psi, or 40 psi. The filter 200j can reach the pressure of about 35 psi within less than 7 minutes (e.g., about 6 min). In some embodiments, the filter 200j can reach a pressure of about 40 psi within about 7 min, 6.5 min, 6 min, 5.5 min, or 5 min.

What is claimed is:

1. A silicon filter for cell mechanoporation, comprising:
a silicon filtering surface;
a plurality of pores extending through the silicon filtering surface and configured to perturb cell membranes as a cell mixture passes through the plurality of pores; and
a support structure disposed on a side of the silicon filtering surface and covering at least 1% of the silicon filtering surface.

2. The silicon filter of claim 1, wherein the silicon filter is configured to receive a volumetric flow rate between 0.5-500 mL/min per mm² porous surface area.

3. The silicon filter of claim 1, wherein the silicon filter is configured to receive a constant volumetric flow controlled by at least one of a peristaltic pump, a syringe pump, and a pressurized reservoir.

4. The silicon filter of claim 1, wherein the silicon filter is configured to withstand a pressure between at least 1-50 psi.

5. The silicon filter of claim 1, wherein the silicon filter is configured to receive a cell mixture comprising between 1.0×10⁸ and 1.0×10¹² cells.

6. The silicon filter of claim 1, wherein the silicon filter is configured to receive a volume between 1-500 mL.

7. The silicon filter of claim 1, wherein the silicon filter is configured to pass the cell mixture through the plurality of pores in a single run without replacing the silicon filter.

8. The silicon filter of claim 1, wherein the silicon filter is configured to pass all of the cell mixture through the plurality of pores in no more than 30 minutes.

9. The silicon filter of claim 1, wherein the plurality of pores comprises no more than 300,000 pores.

10. The silicon filter of claim 1, wherein a width of each pore of the plurality of pores is between 2 μm and 20 μm.

11. The silicon filter of claim 10, wherein each pore of the plurality of pores comprises a circular cross-section, and the width of the pore is a diameter of the pore.

12. The silicon filter of claim 10, wherein a pitch between each pore of the plurality of pores is between 0.5:1 and 100:1 relative to the width of the pore.

13. The silicon filter of claim 1, wherein each pore in the plurality of pores extends linearly through the silicon filtering surface.

14. The silicon filter of claim 1, wherein a thickness of the silicon filtering surface is between 0.1 μm and 100 μm.

15. The silicon filter of claim 1, wherein a thickness of the support structure is between 20 μm and 1 mm.

16. The silicon filter of claim 1, wherein a width of the silicon filtering surface is between 1 mm and 10 cm.

17. The silicon filter of claim 16, wherein the silicon filtering surface comprises a circular shape, and the width is a diameter of the silicon filtering surface.

18. The silicon filter of claim 16, wherein a width of the support structure is at least 30% of the width of the silicon filtering surface.

19. The silicon filter of claim 1, wherein a height of the support structure is between 20 μm and 1 mm.

20. The silicon filter of claim 1, wherein the support structure comprises one or more supporting members disposed on the silicon filtering surface and extending in one or more directions.

21. The silicon filter of claim 20, wherein the support structure comprises a plurality of supporting members disposed on the silicon filtering surface, the plurality of supporting members together forming at least one cross shape separating a plurality of filtering windows in the silicon filtering surface.

22. The silicon filter of claim 20, wherein the support structure comprises a plurality of supporting members disposed independently as stripes on the silicon filtering surface.

23. The silicon filter of claim 1, comprising an oxide layer disposed on the silicon filtering surface between the silicon filtering surface and the support structure.

24. The silicon filter of claim 1, wherein the silicon filtering surface comprises silicon, silicon oxide, silicon nitride, and/or silicon carbide.

25. The silicon filter of claim 1, wherein the support structure comprises silicon, silicon dioxide, silicon nitride, or silicon carbide.

26. The silicon filter of claim 1, wherein the silicon filter is fabricated from a silicon wafer doped with boron, gallium, or phosphorous.

27. The silicon filter of claim 1, wherein the silicon filter is coated with at least one of gold, silver, platinum, Teflon, polyvinylpyrrolidone, an adhesive, and a surfactant.

28. A system, comprising:
the silicon filter of claim 1; and
a cell.

29. The system of claim 28, wherein the cell is a somatic cell, an immortalized cell, a stem cell, or a derivative thereof.

30. The system of claim 28, wherein the cell is a peripheral blood mononuclear cell (PBMC), or a derivative thereof.

31. The system of claim 28, wherein the cell is an immune cell.

32. The system of claim 28, wherein the cell is a T cell, natural killer (NK) cell, monocyte, B cell, or dendritic cell.

33. The system of claim 28, wherein the cell is a stem cell.

34. The system of claim 28, wherein the cell is a human stem cell.

35. The system of claim 28, wherein the cell is an induced pluripotent stem cell, a hematopoietic cell, or a mesenchymal cell.

36. The system of claim 28, wherein the cell is obtained, or derived, from an individual.

37. The system of claim 36, wherein the individual is a human.

38. A method for intracellular delivery of a payload, comprising:
passing a cell mixture comprising cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface and covering at least 1% of the silicon filtering surface; and
collecting a perturbed cell mixture comprising the cells with the payload in the cells.

39. A cell mechanoporation system, comprising:
a filter holder fluidly connectable to a cell mixture source;
one or more silicon cell mechanoporation filters disposed in the filter holder, the one or more silicon cell mechanoporation filters comprising:
a silicon filtering surface;
a plurality of pores extending through the silicon filtering surface and configured to perturb cell membranes as a cell mixture from the cell mixture source passes through the plurality of pores; and
a support structure disposed on a side of the silicon filtering surface and covering at least 1% of the silicon filtering surface;
an output reservoir fluidly connected to the filter holder to collect a perturbed cell mixture; and
a pump configured to move the cell mixture through the one or more silicon cell mechanoporation filters and into the output reservoir.

40. A method of fabricating a silicon cell mechanoporation filter, comprising:
creating a plurality of pores in a filtering layer of a silicon wafer, the silicon wafer comprising a support layer, an oxide layer on a side of the support layer, and the filtering layer on a side of the oxide layer opposite the support layer;
creating a support structure in the support layer of the silicon wafer, wherein the support structure covers at least 1% of the filtering layer; and
removing at least a portion of the oxide layer to expose the plurality of pores in the filtering layer and create one or more silicon cell mechanoporation filters.

41. A method for intracellular delivery of a payload, comprising:
passing a cell mixture comprising at least 1.0×10⁸ cells and the payload through a plurality of pores extending through a silicon filtering surface of a silicon cell mechanoporation filter to perturb the cell mixture, wherein the silicon cell mechanoporation filter comprises a support structure disposed on a side of the silicon filtering surface that covers at least a portion of the silicon filtering surface; and
collecting a perturbed cell mixture comprising the cells with the payload in the cells.

42. The silicon filter of claim 5, wherein the volume is such that a residence time of the cell mixture after passing through the plurality of pores is less than 30 seconds.

43. The silicon filter of claim 2, wherein a pressure gradient across the silicon filtering surface at the volumetric flow rate is less than 20 psi.
